(19) United States
(12) Patent Application Publication
(10) Pub. No.: US 2022/0008382 A1
(43) Pub. Date: Jan. 13, 2022

(54) ALCOHOL ANTIDOTE

(71) Applicant: Evanium Healthcare GmbH, Regensburg (DE)

(72) Inventors: David OFNER, Regensburg (DE); Felix Carlo WERKMANN, Regensburg (DE)

(73) Assignee: Evanium Healthcare GmbH, Regensburg (DE)

(21) Appl. No.: 17/291,522

(22) PCT Filed: Nov. 5, 2019
(86) PCT No.: PCT/EP2019/080288

§ 371 Date: May 5, 2021

(30) Foreign Application Priority Data
Nov. 5, 2018 (EP) 18204386.9

(51) Int. Cl. A61K 31/353; A61K 47/40; A61K 9/14; A61K 47/32; A61K 47/34

(52) U.S. Cl. CPC A61K 31/353; A61K 47/40; A61K 47/34; A61K 47/32; A61K 9/146

(57) ABSTRACT

The present invention relates to the prevention and treatment of alcoholism, alcohol intoxication, and consequential symptoms and diseases associated with alcohol consumption, using specific flavonoids.

The present invention relates to the prevention and treatment of alcoholism, alcohol intoxication, and consequential symptoms and diseases associated with alcohol consumption, using specific flavonoids.

Alcohol intoxication and the associated detrimental effects and side effects the next day are a widespread problem that is difficult to tackle. Since no effective antidote is available for alcohol, by far the most commonly consumed poison, medical treatment of alcohol-related symptoms is very difficult or even impossible.

This is due, inter alia, to the complex mechanism of action underlying drinking alcohol. In contrast to benzodiazepines, for example, alcohol (ethanol), as a very small molecule, is able to develop its effect at various binding sites of the responsible receptor. Above all, the GABA_{A} receptor is responsible for the majority of the effects of alcohol. This ionotropic receptor comprises five subunits (two α, two β, one γ/δ/ε/θ/π), wherein tonic receptors, which comprise one δ subunit in combination with two α4 or α6 and two β3 subunits, are particularly sensitive to ethanol¹. If ethanol binds to the receptor between the β subunit and the a subunit, the efficiency of the orthosteric agonist GABA increases. This leads to an increased influx of negatively charged chloride ions at the postsynapse, which significantly inhibits the transmission of electrical impulses at the synapse. This leads to a feeling of intoxication, but if the dosage is too high, severe side effects such as coordination disorders or respiratory paralysis can occur. There are two binding sites: one between the α⁺/β⁻ interface (high affinity from 3 mM concentration) and one between the β⁺/α⁻ interface at the amino acid β-265ASN (low affinity from 50 mM)².

It was discovered by chance that the compound Ro15-4513, which is actually synthesized as a benzodiazepine antidote and is an inverse agonist at the GABA_{A} receptor, is less effective in benzodiazepine overdoses, but is all the more effective in alcohol intoxication.

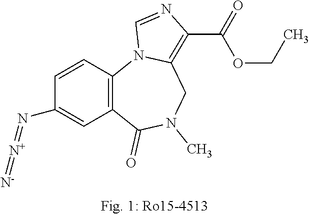 [SMILES: CCOC(=O)c1ncn2c1CN(C)C(=O)c1cc(N=[N+]=[N-])ccc1-2]

This is due to the fact that Ro15-4513, like alcohol, docks to the α⁺/β⁻ interface of the ethanol-sensitive α4β3δ and α6β3δ GABA_{A} receptor and the azide group at C7 blocks the binding site for alcohol³. This is possible because the binding site for alcohol is very similar to the binding site for the benzodiazepines at the α/γ interface³.

As a result, further derivatives with other functional groups were synthesized at C7, and all of them are effective against alcohol, but have some significant disadvantages that make clinical use impossible. All of the compounds have a very short half-life (approx. 30 min) and must be administered intravenously because of their very low bioavailability. This makes practical application both impossible to carry out and very dangerous for the patient, because it can happen that the antidote loses its effect after a short time and the patient suddenly becomes drunk again. In addition, the active ingredient can only antagonize comparatively low doses of alcohol (up to approx. 2 per mille) at the high-affinity binding site, since the low-affinity binding site is occupied from 2 per mille².

Finally, there is a crucial problem with all of the compounds which ultimately makes use thereof as an antidote impossible. Since the substances were originally synthesized as benzodiazepine antidotes, they thus also act as inverse agonists on the phasic GABA_{A} receptors with a gamma subunit and are not at all effect-specific². As a result, if the dosage is minimally incorrect, dangerous side effects such as epileptic seizures or convulsions can occur due to the unspecific blockade of the GABA receptors.

The underlying object of the present invention was therefore to circumvent the above problems and provide an effective alcohol antidote. To this end, GABA_{A} modulators which act subunit-specifically on α4β3δ and α6β3δ receptors were specifically sought.

The well-known hypnotic agents methaqualone and etomidate as well as the anticonvulsant loreclezole bind to the β(+)/α(−) interface of the GABA_{A} receptor on the amino acid β-265ASN of the low-affinity binding site for drinking alcohol. They function as positive allosteric modulators (PAM) in that they change the conformation of the orthosteric GABA side so that the effect of GABA is enhanced⁴. It is interesting here that the three active ingredients differ fundamentally in their mechanism of action from the benzodiazepines and also bind to tonic GABA_{A} receptors with a δ subunit. Because of these properties, these active ingredients were very interesting as lead compounds. A comparison of the structural formulae of the three active ingredients shows several similarities:

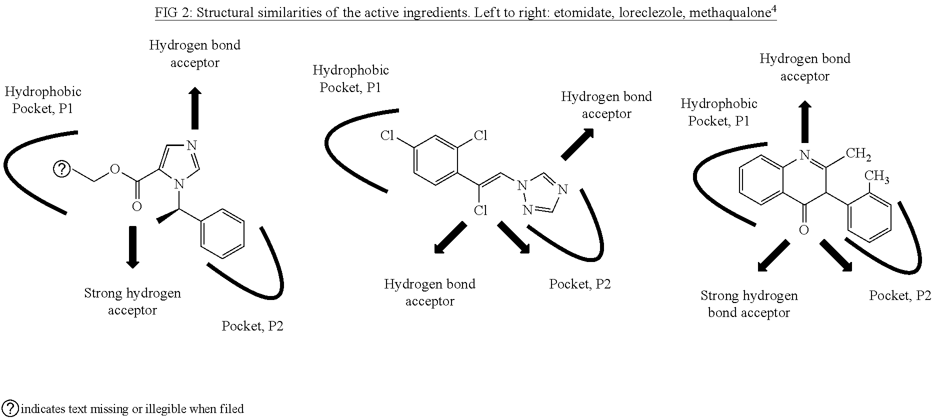 [SMILES: CCOC(=O)c1cncn1[C@H](C)c1ccccc1 ; Cl/C(=C\n1cncn1)c1ccc(Cl)cc1Cl ; CC1=Nc2ccccc2C(=O)C1c1ccccc1C]

These structural similarities can also be found in another class of active ingredients, specifically in the flavonoids.

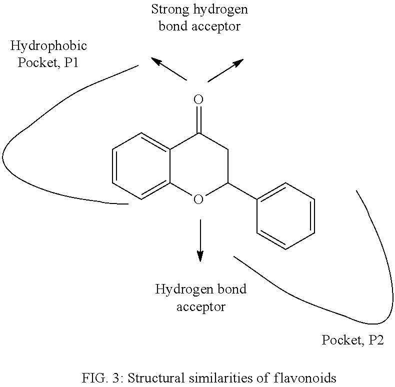 [SMILES: O=C1CC(c2ccccc2)Oc2ccccc21]

Flavonoids are a group of secondary plant substances which are formally derived from the basic body flavan. There are around 8000 compounds in nature, the diversity of which arises from different oxidation stages in the oxygen-containing ring, different substitutions on the aromatic rings, and the addition of sugars (glycoside formation). Moreover, flavonoids are found in a large number of plants and therefore also in human food. Some of these plant components have health-promoting properties, which is why this group of substances is of particular medical interest.

In the present invention it has now surprisingly been found that certain flavonoids are able to act in a subunit-specific manner on α4β36 and α6β36 GABA_{A} receptors as negative allosteric modulators. Depending on the subunit, they prevent the binding of GABA, so that ethanol also has no effect. These flavonoids are based on the basic structure of (+)taxifolin, wherein certain radicals are possible at the various positions of the ring system.

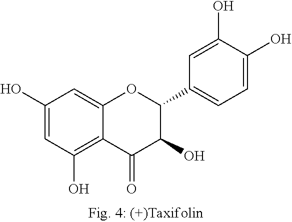 [SMILES: O=C1c2c(O)cc(O)cc2O[C@H](c2ccc(O)c(O)c2)[C@H]1O]

It has been found that the binding pocket of taxifolin and other compounds of the following formula (I) is the β(+)/α(−) interface on the amino acid β-265ASN (low-affinity binding site of ethanol), so that alcohol can be counteracted on different levels:
up to 2 per mille using subunit-specific, negative modulation
from 2 per mille using direct, competitive inhibition of the alcohol in the low-affinity binding pocket.

A first aspect of the invention therefore relates to a flavonoid of the general formula (I)

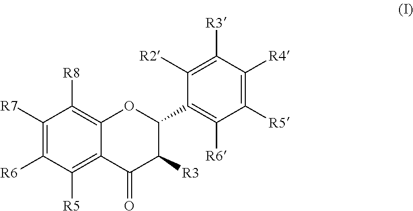

wherein:
R7, R4′=—OH, C₁₋₁₈-alkoxy, C₃₋₁₀-cycloalkoxy, C₁₋₁₈-alkenyloxy, C₃₋₁₀-cycloalkenyloxy, C₁₋₁₈-hydroxyalkoxy, mono- or oligoglycosyl, ester (e.g., succinate);
R5, R3, R3′=—H—OH, C₁₋₁₈-alkoxy, C₃₋₁₀-cycloalkoxy, C₁₋₁₈-alkenyloxy, C₃₋₁₀-cycloalkenyloxy, C₁₋₁₈-hydroxyalkoxy, mono- or oligoglycosyl, ester (e.g. succinate);
R6, R8, R2′, R5′, R6′=—H or C₁₋₈ alkyl, C₃₋₈-cycloalkyl, C₃₋₁₀-alkenyl, or C₃₋₁₀-cycloalkenyl;

for use in the prevention and/or treatment of alcoholism, alcohol intoxication, and consequential symptoms and diseases associated with alcohol consumption.

As an alcohol antidote, the flavonoids used according to the invention have significant advantages over earlier compounds such as, e.g. Ro-15-4513, since, on the one hand, due to the specific binding no side effects occur, even at high doses (NOAEL taxifolin level 1500 mg/kg), and, on the other hand, the alcohol can be counteracted by competitive inhibition, even at high dosages.

In this way it is possible to treat alcohol intoxication, since the effect of ethanol on the CNS is blocked by flavonoids of the general formula (I). This is particularly important in order to counteract alcohol-related behavioural disorders, e.g. aggressiveness, or the failure of vital functions in the context of emergency treatment. Intravenous administration is also suitable for this purpose.

On the other hand, it is also possible to specifically increase the receptor density of ethanol-sensitive GABA receptors. This plays a major role in the treatment and prevention of alcohol addiction. This can help alcoholic patients on the one hand to get out of addiction more quickly, and, on the other hand, long-term medication can reduce the risk of relapse, as the alcohol no longer has any effect.

Moreover, the flavonoid of the general formula (I) is also able to alleviate the acute side effects of excessive alcohol consumption, since the symptoms the next day can be explained at least in part by a reduction in the GABA_{A} receptor density and the withdrawal symptoms triggered thereby. This is particularly relevant since tonic GABA_{A} receptors with a δ subunit are very susceptible to downregulation by endocytosis. Significant internalization of tonic GABA_{A} receptors was demonstrated both in vitro and in vivo even after a single dose of alcohol⁵.

Finally, the flavonoid of the general formula (I) is also able to prevent secondary diseases, in particular impairments of the nervous system as a result of alcohol consumption. This can also be explained by its effect as a negative, allosteric modulator of tonic GABA_{A} receptors. This is because alcohol-related neurotoxicity can be explained, inter alia, by the upregulation of certain excitatory glutamate receptors, in particular the NMDA receptor⁶. At the same time, there is a downregulation of inhibitory GABA_{A} receptors with a δ subunit. If the ethanol concentration is now reduced, the nerve cell is overloaded with stimuli, which ends in apoptosis of the cell (excitotoxicity). Compounds of the general formula (I) can counteract the downregulation of tonic GABA_{A} receptors and increase the receptor density of these inhibitory receptors. This can prevent excitatory neurotoxicity as a result of alcohol consumption.

A combination of a flavonoid of the general formula (I) and vitamins, in particular thiamine, and its pharmaceutically acceptable salts, derivatives and prodrugs, can be used to prevent neurological damage due to an alcohol-related nutrient deficiency, e.g. in the case of Wernicke encephalopathy.

Taxifolin and other compounds of the formula (I) are distinguished by a basic structure with a single bond between positions 2 and 3. As a result, the flavonoid loses its planarity and one or two centres of chirality (at 2 and at 3) result (depending on the substitution at R3). Only the (2S) isomers (if only one centre of chirality is present) or the (2R, 3R) trans isomers (with two centres of chirality) are suitable for use according to the invention, since only these can assume the correct position in the binding pocket. The docking to the β(+)/α(−) interface of the GABA receptor, which is responsible for a specific alcohol-antagonistic effect, takes place stereospecifically.

In contrast thereto, flavonoids with a 2,3 double bond, such as, e.g., quercetin, morin, apigenin, luteolin, chrysin and baicalein, have a planar structure and exhibit an action similar to benzodiazepine. Such flavonoids are the main active ingredients of calming plant extracts such as St. John's wort and passion flower and have therefore been used for centuries as herbal remedies for insomnia, internal distress, and anxiety.

Further essential features of the inventive flavonoids according to formula (I) are an oxane ring and a keto group at position 4. These groups act as hydrogen-bond acceptors and thus stabilize the position of the flavonoid in the binding pocket of the receptor.

Certain substituents are present at different ring positions of the flavonoid skeleton according to formula (I). The radicals R7 and R4′ are selected from the group comprising OH, C₁₋₁₈-alkoxy, C₃₋₁₀-cycloalkoxy, C₁₋₁₈-alkenyloxy, C₃₋₁₀-cycloalkenyloxy, C₁₋₁₈-hydroxyalkoxy, mono- or oligoglycosyl, and ester (e.g. succinate). OH is preferred.

The radicals R5, R3, and R3′ are selected from the group consisting of H, OH, C₁₋₁₈-alkoxy, C₃₋₁₀-cycloalkoxy, C₁₋₁₈-alkenyloxy, C₃₋₁₀-cycloalkenyloxy, C₁₋₁₈-hydroxyalkoxy, mono- or oligoglycosyl, and ester (e.g. succinate). H and OH are preferred.

The radicals R6, R8, R2′, R5′, and R6′ are selected from H or C₁₋₈-alkyl, C₃₋₈-cycloalkyl, C₃₋₁₀-alkenyl, and C₃₋₁₀-cycloalkenyl. H and C₁₋₈-alkyl are preferred.

Preferred are flavonoids of the general formula (I), wherein:

R7 and R4′ are each OH,

R5, R3, and R3′ are each H or OH, and

R6, R8, R2′, R5′, and R6′ are each H or C₁₋₈ alkyl, preferably H.

Preferred are flavonoids of the general formula (I), wherein:

R2′, R5′, R6′, R6, and R8 are each H, and

R3′, R4′, R3, R5, and R7 are each OH.

As used herein, the term “alkyl” refers to a straight or branched chain hydrocarbon group. The term “C₁₋₈ alkyl” refers to a C₁₋₈ alkyl chain. Examples of alkyl groups are methyl, ethyl, n-propyl, isopropyl, tert-butyl, and n-pentyl. Alkyl groups can optionally be substituted with one or more substituents.

The term “cycloalkyl” refers to a monocyclic or bicyclic ring system with at least one saturated ring. Cycloalkyl groups can optionally be substituted with one or more substituents. In one embodiment, 0, 1, 2, 3, or 4 atoms of each ring of a cycloalkyl group can be substituted with a substituent. Representative examples of cycloalkyl groups are cyclopropyl, cyclopentyl, cyclohexyl, cyclobutyl, cycloheptyl, cyclopentenyl, cyclopentadienyl, cyclohexenyl, cyclohexadienyl, and the like.

The term “alkenyl” refers to an unsaturated hydrocarbon chain, which can be a straight or branched chain, containing at least one carbon-carbon double bond. Alkenyl groups can optionally be substituted with one or more substituents.

The term “cycloalkenyl” refers to a monocyclic or bicyclic ring system with at least one non-aromatic ring that has at least one carbon-carbon double bond. Cycloalkenyl groups can optionally be substituted with one or more substituents.

The term “(cyclo)alkoxy” refers to a —O-(cyclo) alkyl radical, wherein (cyclo)alkyl is as defined above.

The term “(cyclo)alkenyloxy” refers to the group —O(cyclo)alkenyl, wherein (cyclo)alkenyl is as defined above.

The term “hydroxyalkoxy” denotes a —O-alkyl radical, wherein one or more hydroxy groups are bonded to primary or secondary carbon atoms of the alkyl radical.

The compounds of the general formula (I) can be used in the form of pharmaceutically acceptable salts, derivatives, or prodrugs, in particular with glycosyl, ether, or ester groups at the positions of OH groups. These derivatives are converted back to the main active ingredient by enzymatic cleavage in the body.

Polyphenols such as the compounds of the general formula (I) generally have a low bioavailability, which can be explained in particular by low solubility in water, low stability, and pronounced metabolism by phase II enzymes. The first two problems according to the present invention can be solved using a suitable formulation as described hereinafter, but the conversion to prodrugs is also a very elegant way to circumvent the low bioavailability of the flavonoids. Here, on the one hand, solubility in water can be increased; on the other hand, it is possible to protect the phenolic hydroxyl groups from oxidation or biotransformation. Finally, permeability can also be positively influenced using conversion to prodrugs.

The position of the hydroxyl group, which is to be protected by a prodrug approach, is decisive for this. In general, certain OH positions are more susceptible to metabolism by phase II enzymes, wherein in particular glucuronidation by UDP-glucuronosyltransferases (UGT), sulfonation by sulfotransferases (SULT), and O-methylation by the enzyme catechol-O-methyltransferase (COMT) stand in the foreground.

In principle, the hydroxyl group at position R5 is inert due to the interaction with the adjacent carbonyl group at R4 and is therefore protected. The OH groups on R7, R3, R4′, and R3′, on the other hand, are reactive, which is why derivatization of these groups appears sensible.

The hydroxyl groups at R7, R3, R4′, and R3′ are transformed by both UGT and SULT. Since COMT, on the other hand, only methylates catechol groups, only the hydroxyl groups on ring B are affected thereby, and only if a catechol group is present. In particular, the OH group is then methylated at R3′. However, since O-methylation now leads to more lipophilic products with higher permeability through the blood-brain barrier, metabolization by COMT is more advantageous than transformation by UGT or SULT. In the latter, highly polar connections develop, which cross the blood-brain barrier with difficulty.

For this reason, the following applies for the priority of derivatizing the hydroxy groups for conversion to prodrugs:

R7=R3>R3′=R4′>R5

There are different approaches for derivatizing these groups. According to the invention, in particular esters (e.g. carbonates, carbamates, sulfamates, phosphates/phosphonates, neutral or anionic carboxylic acid esters, and amino acid esters), ethers (e.g. alkyl ethers, aryl ethers, and hydroxyalkyl ethers), glycosides (monosaccharides and oligosaccharides) are provided as prodrugs.

According to the invention, “mono- and oligoglycosyl radicals” preferably include hexosyl radicals, in particular ramnosyl radicals and glucosyl radicals. Further examples of suitable hexosyl radicals are allosyl, altrosyl, galactosyl, gulosyl, idosyl, mannosyl and talosyl. Alternatively or in addition, mono- and oligoglycosyl radicals can comprise pentosyl radicals. The glycosyl radicals can be linked α- or β-glycosidically to the base body. A preferred disaccharide is, for example, 6-O-(6-deoxy-α-L-mannopyranosyl)-β-D-glucopyranoside.

It is also possible to convert the phenolic hydroxyl group to a hemiacetal with various aldehydes (e.g. acetaldehyde). The hydroxyl group of this hemiacetal can now be derivatized just like the phenolic hydroxyl group. An example of this are the phosphonooxy alkyl prodrugs.

Bioavailability can also be improved by combining with inhibitors of the phase II enzymes of biotransformation, e.g., piperine, protease inhibitors such as Atazanavir, antifungal drugs such as ketoconazole, opioid receptor antagonists such as nalmefene and naltrexone, as well as various polyphenols. However, due to possible drug interactions, this is the most unsuitable method; in addition, many inhibitors have their own pharmacological profile.

According to the present invention, flavonoids of the general formula (I) can be used for the prevention and/or treatment of alcoholism, alcohol intoxication, and consequential symptoms and diseases associated with alcohol consumption.

As used herein, the term “alcoholism” includes physical and/or psychological dependence on alcohol (addiction syndrome). It has been found that administering flavonoids of the formula (I) can counteract the development of an addiction syndrome and can thus be used to prevent alcoholism. If alcoholism is already present, it is possible to provide treatment, including alcohol dishabituation and/or withdrawal from alcohol, using flavonoids of the formula (I).

Withdrawal symptoms can occur when alcohol consumption is reduced or stopped suddenly. Withdrawal symptoms include nausea, nervousness, difficulty sleeping, an urge to drink alcohol, irritability, and depression. As the physical addiction progresses, sweating, tremors, flu-like symptoms, seizures, and hallucinations occur, as well. These and other withdrawal systems can be prevented or mitigated using the inventive flavonoids of the formula (I).

As used herein, the term “alcohol intoxication” encompasses all stages of acute alcohol intoxication. Depending on blood alcohol concentration, a distinction is made between the stages of excitation (0.2-2.0%.), hypnosis (2.0-2.5%.), narcosis (2.5-4.0%.), and asphyxia (over 4.0%.). Due to their specific binding to the α4β36 or α6β36 GABA_{A} receptor, flavonoids of the formula (I), as an allosteric modulator, are able to counteract the binding of alcohol to the GABA_{A} receptor, so that alcohol has no effect.

In addition to the prevention and treatment of acute alcohol intoxication, according to the invention using flavonoids of the general formula (I) can also prevent and or treat associated consequential symptoms and prevent associated diseases. Such associated diseases are diseases that can be attributed to long-term alcohol abuse, such as, in particular, impairment of the nervous system (through destruction of axons such as the myelin sheaths of the brain and the peripheral nervous system, e.g. neuropsychological weaknesses, memory disorders, impaired consciousness, dementia syndrome, neuropathic pain etc.).

The consequential symptoms associated with alcohol consumption also include acute consequences, such as, in particular, hangovers. A hangover is understood here as the malaise and impairment of physical and mental performance as a result of excessive alcohol consumption. A hangover mainly includes the symptoms of headache, stomach pain, nausea and vomiting, concentration disorders, increased tendency to sweat, stomach and muscle pain, depressive mood, and general malaise on the following days, especially on the day after the excessive alcohol consumption.

Using flavonoids of the general formula (I), it is possible according to the invention to reduce the frequency of alcohol consumption compared to the frequency before the treatment. It is also possible to reduce the amount of alcohol. It also possible to increase the rate of abstinence.

In order to achieve good effectiveness, the flavonoids of the general formula (I) are preferably administered in a form in which they have good bioavailability. In this connection, the sometimes low solubility of the flavonoids of the general formula (I) in water, which affects their bioavailability, is problematic. It was therefore a further aim of the present invention to improve solubility and to provide the flavonoids of the formula (I) in a form in which they are more soluble in water and can be better absorbed in the human organism.

In principle, different methods are used in this field to increase the solubility of pharmaceutically active compounds in water. In the present invention it has now been found, specifically for the flavonoids of the formula (I), that many of the otherwise customary methods are not suitable here. No significant improvement in solubility could be achieved through the formulation of solid dispersions with typical pharmaceutical polymers such as, e.g., polyvinylpyrrolidone (PVP), polyvinylpyrrolidone-vinyl acetate copolymer, Soluplus®, polyacrylic acids, or various biopolymers such as hydroxypropyl methylcellulose, hydroxypropyl cellulose, methylcellulose, sodium carboxymethylcellulose, maltodextrin, shellac, collagen hydroloysate, chitosan, gellan, xanthan, or alginate.

In addition, no adequate improvement in physicochemical properties could be observed through the formulation of co-crystals with urea, caffeine and nicotinamide, through the formulation of micelles with various surfactants such as lecithin, polysorbate 80, vitamin E TPGS, macrogol 15-hydroxystearate, macrogol glycerol hydroxystearate, or sodium dodecyl sulfate. or through the micronization of the flavonoid particles.

Surprisingly, however, through complexing with cyclodextrins the flavonoids can be converted into readily water-soluble inclusion complexes with excellent bioavailability. For use according to the invention, the above flavonoids are therefore preferably used in the form of a complex of the general formula (II),

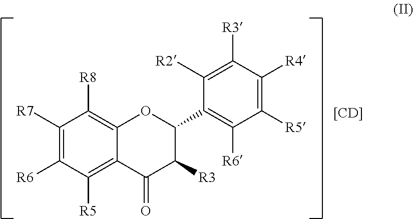

wherein the radicals R2′, R6′, R3, and R5-R8 are defined as above in connection with flavonoids of the formula (I) and CD is a cyclodextrin molecule or a derivative thereof.

Cyclodextrins are a class of cyclic oligosaccharides that are composed of α-1,4-glycosidically linked glucose molecules. The cyclodextrins are named differently depending on the number of glucose units building them, wherein α-cyclodextrin contains 6 glucose molecules, β-cyclodextrin contains 7 glucose molecules, γ-cyclodextrin 8 contains glucose molecules, and δ-cyclodextrin contains 9 glucose molecules. According to the invention, in particular an α-, β-, or γ-cyclodextrin, preferably β- or γ-cyclodextrin, can be used as the cyclodextrin.

In inclusion complexes with cyclodextrins, the crystalline structure is dissolved and each molecule is individually “encapsulated” in a cyclic starch ring. On the one hand, this results in advantages similar to those of a solid dispersion (amorphous form, larger surface, etc.); on the other hand, the solubility of the flavonoid itself is increased, since the outside of the cyclodextrin is very hydrophilic and thus acts as a “Trojan horse”. In all of the cyclodextrin complexes examined, solubility was significantly higher than that of taxifolin. Finally, CD encapsulation can also increase the stability of the active ingredient, which is particularly advantageous for sensitive flavonoids. The latter tend to oxidize during processing or in the GI tract, which would render them ineffective. The type of cyclodextrin (α, β, γ, or δ), which differ mainly in the ring diameter, and the exact production method of the cyclodextrin/flavonoid complexes make a big difference in the quality of the complex compound.

Cyclodextrins can be present in underivatized form or in derivatized form in which, for example, one or more hydroxyl groups of glucose units carry substituents. For example, the C₆ carbon atom on one or more glucose units of the cyclodextrin can be alkoxylated or hydroxyalkylated. For example, the hydrogen atom of the hydroxyl group on the C₆ carbon atom of one or more glucose units can be replaced by C1-18-alkyl or C₁₋₁₈-hydroxyalkyl groups. Particularly preferred are in particular 2,6-di-O-methyl-cyclodextrin and 2-hydroxypropyl-cyclodextrin. In addition, sulfoalkyl cyclodextrins, especially sulfoethyl, sulfopropyl and sulfobutyl cyclodextrins are of interest.

Using cyclodextrins it is possible according to the invention not only to increase the solubility of the flavonoids, but also to significantly improve their biological effectiveness. Accordingly, complexes of the general formula (II) are particularly suitable for use in the prevention and/or treatment of alcoholism, alcohol intoxication, and consequential symptoms and diseases associated with alcohol consumption, as defined above.

However, some active ingredient complexes (in particular γ-CD complexes) exhibit a special dissolution behaviour, a so-called “spring” profile. These active ingredient CD complexes initially dissolve very well in an aqueous medium and a high active ingredient concentration is achieved. But after a certain time the active ingredient recrystallizes and the concentration drops rapidly. To counteract this behaviour, in one preferred embodiment of the invention a water-soluble polymer can be integrated into the complex or in solution as a “parachute”, effectively preventing recrystallization of the active ingredient and thus maintaining the high initial concentration for a long time. Very low polymer concentrations are often sufficient for achieving the desired effect. One aspect of the invention accordingly relates to a ternary complex made of a flavonoid of the general formula (I), a cyclodextrin, and a water-soluble polymer. The water-soluble polymer is contained in solution preferably in an amount of at least 0.0025% w/v, in particular 0.0025-1.0% w/v, more preferably 0.025-0.5% w/v, for example 0.25% w/v. Based on the flavonoid of the general formula (I), the polymer:flavonoid mass ratio is preferably between 1:0.5 and 1:80, in particular between 1:3 and 1:15. In practice, mass ratios in the range between 1:6 and 1:8 have proven to be optimal.

Examples of water-soluble polymers which are particularly suitable according to the invention are polyethylene glycol, e.g. PEG 6000, polyvinyl alcohol, poloxamer, e.g. Poloxamer 188, and mixtures thereof, such as e.g. mixtures of PEG and PVA (Kollicoat® IR). These polymers are built up from ethylene oxide blocks and exhibit very promising properties. The interactions with the hydroxyl groups of the flavonoid are not so strong that precipitation occurs; at the same time, the polymers also interact with the hydroxyl groups of the cyclodextrin. This increases complex stability.

The increase in complex stability can be explained by the fact that the polymer interacts with the active ingredient and the cyclodextrin and thus stabilizes the active ingredient in the CD cavity. This must be taken into account when selecting the right polymer, because if the interaction with the active ingredient is too strong, the polymer-active ingredient complex flocculates and Ks drops. If the interaction with the cyclodextrin is too strong, the polymer and active ingredient compete for the CD cavity and Ks drops in this case, as well. Finally, it must be ensured that the polymer does not increase or only slightly increases the viscosity of the solution, since otherwise the CD complex formation is made more difficult.

Another possibility for improving the solubility of flavonoids of the formula (I) is, according to the invention, to form a solid dispersion with basic polymers or copolymers of methacrylic acid and/or methacrylate. It was found that in particular Eudragit®E in combination with flavonoids of the general formula (I) leads to a solid dispersion with good water solubility and in this way high bioavailability of the flavonoid can be achieved.

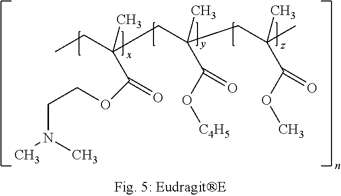

The observed improvement in solubility is due to the intermolecular interactions between the carbonyl group of the methacrylic ester and the hydroxyl groups (or similar groups) of the flavonoid of formula (I). This stabilizes the flavonoid in its amorphous form, which considerably improves its solubility in water. In contrast to other polymers, such as, e.g., PVP, the aminoalkyl groups of Eudragit, which are cationic in the protonated state, make the polymer water-soluble, even if it interacts strongly with the flavonoid.

For the inventive use, in one preferred embodiment the above flavonoids are therefore used as a solid dispersion with basic polymers or copolymers of methacrylic acid and/or methacrylate.

A further subject matter of the invention therefore relates to a solid dispersion of a flavonoid of the general formula (I) and a (co)polymer of methacrylic acid and/or methacrylate such as, e.g., Eudragit®E, Eudraguard®protect, or Kollicoat®Smartseal.

For administration, flavonoids of the formula (I), cyclodextrin complexes of the formula (II), ternary complexes with water-soluble polymers or solid dispersions with (co)polymers of methacrylic acid and/or methacrylate, can be present, for example, as a pharmaceutical formulation in the form of tablets, capsules, pills, coated tablets, granules, suppositories, pellets, solutions, or dispersions, wherein the active ingredient can optionally be combined with pharmaceutically acceptable adjuvants and excipients. Such pharmaceutical formulations can be produced in a customary manner familiar to the person skilled in the art.

Administration can in principle take place in any desired way, with oral administration being preferred. In addition, intravenous administration can also be indicated, in particular for the treatment of alcohol intoxication.

Solid formulations for oral administration can contain not only the active ingredient, but also customary adjuvants and excipients, such as diluents, e.g., lactose, dextrose, sucrose, cellulose, corn starch, or potato starch; lubricants, e.g., silicate, talc, stearic acid, magnesium or calcium stearate, and/or polyethylene glycols; binding agents, e.g. starches, gum arabic, gelatin, methyl cellulose, carboxymethyl cellulose, or polyvinylpyrrolidone; disintegrants, e.g., starch, alginic acid, alginates or sodium starch glycolates, foaming mixtures; dyes; sweeteners; wetting agents such as lecithin, polysorbates, lauryl sulphates; as well as other customary formulation adjuvants.

Liquid formulations for oral administration can be, for example, dispersions, syrups, emulsions, and suspensions. A syrup can contain, e.g., sucrose or sucrose with glycerol and/or mannitol and/or sorbitol as a carrier. Suspensions and emulsions can contain, e.g., a natural resin, agar, sodium alginate, pectin, methyl cellulose, carboxymethyl cellulose, or polyvinyl alcohol as a carrier.

Solutions for intravenous injection or infusion can contain, e.g., sterile water as a carrier or they can preferably be in the form of sterile, aqueous, isotonic saline solutions.

A further subject matter of the invention is therefore a pharmaceutical composition for oral administration, comprising a complex of the general formula (II), a ternary complex with a water-soluble polymer, or a solid dispersion as described above. Moreover, the pharmaceutical composition can comprise one or more pharmacologically acceptable adjuvants and/or carriers.

Suitable dosages of a flavonoid of the general formula (I) in an inventive pharmaceutical composition for oral administration can be in the range of about 25 mg to 1200 mg. Dosages of 150 to 600 mg, in particular 300 mg to 450 mg, are preferred.

A further subject matter of the invention is a pharmaceutical composition described above for use in the prevention and/or treatment of alcoholism, alcohol intoxication, or consequential symptoms associated with alcohol consumption or in the prevention of consequential diseases associated with alcohol consumption.

The invention shall be further illustrated using the following drawings and examples.

### DRAWINGS

FIG. 1: 1H-NMR spectroscopic examination of taxifolin and various cyclodextrin complexes

FIG. 2: Solubility of various cyclodextrin complexes of taxifolin in water (in mg/ml) depending on the how said complexes are produced and the cyclodextrin used (FIG. 2A: β-cyclodextrin, FIG. 2B: γ-cyclodextrin)

FIG. 3: Results of the DSC analysis of various solid dispersions of taxifolin and Eudragit E in different mixing ratios.

FIG. 3A: Thermogram for Eudragit E CDE 1:1+reference

FIG. 3B: Thermogram for Eudragit E CDE 2:1+reference

FIG. 3C: Thermogram for Eudragit E CDE 3:1+reference

FIG. 4: Solubility of solid dispersions with Eudragit E

FIG. 5: Dissolution behaviour of taxifolin compared to a complex with cyclodextrin B and a solid dispersion with Eudragit E (CSE 2:1)

FIG. 6: Hangover symptoms after alcohol consumption when using
A) Taxifolin (raw), B) Taxifolin/ß-CD, C) Taxifolin/ß-CD complex,
D) Ternary complex, E) Taxifolin/Eudragit E (mixture), and,
F) Taxifolin/Eudragit E (solid dispersion)
The following symptoms have been investigated:
(1) General condition
(2) Headache
(3) Nausea
(4) Dizziness
(5) Cognitive performance
(6) Gastrointestinal
(7) Motivation
(8) Fatigue

### EXAMPLES

1. Preliminary Tests for Complex Formation with Cyclodextrins

Initial preliminary tests were undertaken in order to obtain first indications of the optimal parameters for complex production. These initial preliminary tests were carried out with β-CD, 2-hydroxypropyl-β-CD, and γ-CD, which, due to their ring size, are best suited for forming an inclusion complex with flavonoids.

First, a suitable quantitative detection method for taxifolin was developed. The complex formation in aqueous solution with the various cyclodextrins was then detected using a 1H-NMR analysis. Finally, the increased stability of the taxifolin/cyclodextrin complexes in aqueous solution was demonstrated and quantified. Based on the preliminary tests, the γ-cyclodextrin could be determined from the types of cyclodextrin as the optimal cyclodextrin for complex formation with taxifolin.

¹H-NMR spectroscopy was used to provide qualitative evidence of complex formation in aqueous solution. In this way, the characteristic spectra of taxifolin and cyclodextrin can be determined. When a complex is formed, certain signals are shifted. Moreover, the exact three-dimensional structure of the complex and the conformation of the flavonoid in the cyclodextrin cavity can be determined.

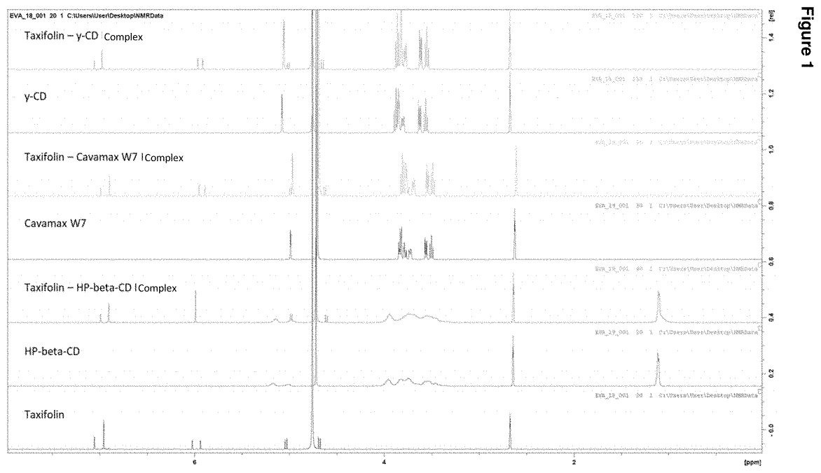

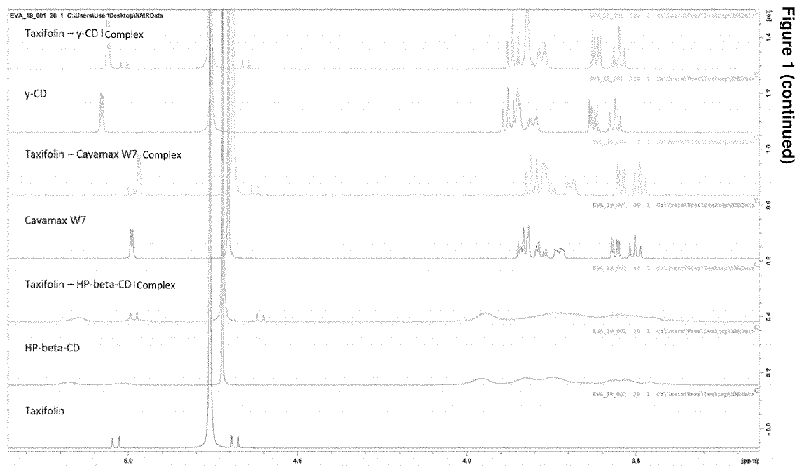

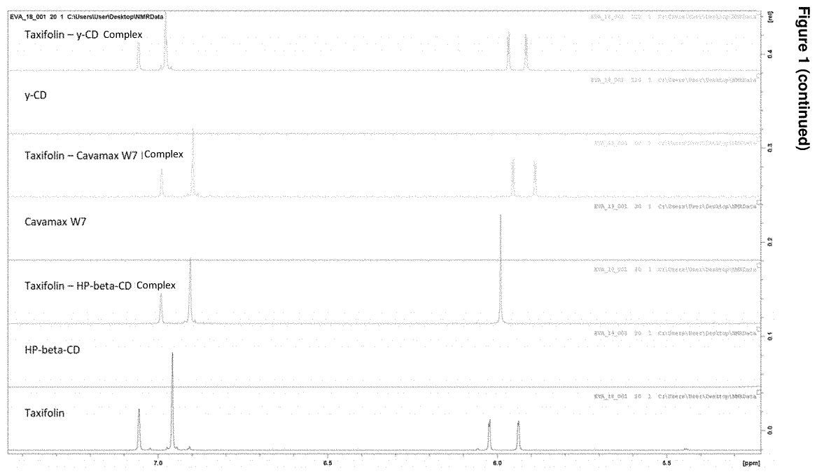

In order to achieve complex formation in solution, taxifolin and the specific cyclodextrin (β, HP-β, or γ) were weighed out in a molar ratio of 1:1, dissolved in D20/DMSO (80/20 v/v), and stirred for 3 hours at room temperature and 600 rpm. The sample was then measured. The reference solutions (taxifolin, β-CD, HP-β-CD, and γ-CD) were only dissolved in D20/DMSO (80/20 v/v) and then measured. The results are shown in FIG. 1.

Discussion: Due to the signal shifts, the results clearly indicate complex formation in solution. However, the results can also be used to precisely predict the position of the flavonoid in the CD cavity. This is because the protons, which exhibit a signal shift due to the complex formation, are embedded in the CD cavity. There are clear differences between β-CD/HP-β-CD, and γ-CD.

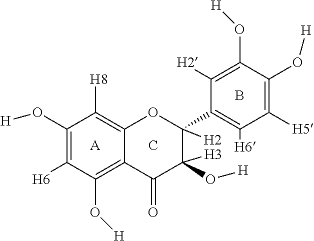

In the case of β-CD and HP-β-CD, the signals of the protons H2′, H5′, and H6′ are shifted, which indicates that ring B is embedded in the CD cavity. This also agrees with the prevailing opinion that, because of their ring size, β-CDs mainly include monocyclic aromatics. Based on 1H-NMR spectroscopy, the following conformation of the flavonoid in the β-CD/HP-β-CD cavity can be predicted:

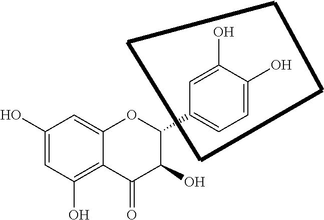 [SMILES: O=C1c2c(O)cc(O)cc2O[C@H](c2ccc(O)c(O)c2)[C@H]1O]

It is interesting, however, that in the HP-β-CD complex the signals of the protons H6 and H8 combine to form a common peak. This is presumably due to hydrogen bonds between the hydroxypropyl radical of the cyclodextrin and various radicals on ring A.

In the case of γ-CD, the signals of protons H6 and H8 in particular, but also, albeit less pronounced, those of protons H2 and H3, are shifted. This indicates that rings A and in part C are embedded in the CD cavity. This is also in agreement with the prevailing opinion that γ-CD mainly includes polycyclic aromatics due to its ring size. The following conformation of the flavonoid in the γ-CD cavity can be predicted based on ¹H-NMR spectroscopy:

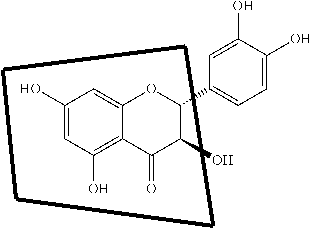 [SMILES: O=C1c2c(O)cc(O)cc2O[C@H](c2ccc(O)c(O)c2)[C@H]1O]

The different position of the flavonoid in the CD cavity naturally has an influence on the solubility and permeation-increasing effect of the cyclodextrin.

In the case of β-CDs, a surfactant-like structure with a hydrophilic head (ring A in the CD cavity) and a hydrophobic tail (ring A/C) is formed. Moreover, the intramolecular hydrogen bonds on the outer ring of the β-CD, which are responsible for the low water solubility (18.5 mg/ml at 25° C.) of the natural β-CD, are broken. This explains why the taxifolin/β-CD complex is even more soluble in water than taxifolin and β-CD alone. Due to the surfactant-like structure and the tendency of the β-CD to extract cholesterol from the cell membrane, a positive influence on the permeation of the flavonoid can also be assumed. Moreover, the catechol group on ring B is particularly prone to oxidation in the GI tract, a process that can be effectively counteracted by encapsulation with β-CD.

In the case of γ-CD, however, rings A and C are included and no surfactant-like structure is formed. Moreover, the unstable catechol group on ring B remains free, so that the stability of the flavonoid is not increased by encapsulation with γ-CD. The natural γ-CD has very high solubility in water (223 mg/ml at 25° C.) because the outer ring is very flexible and thus fewer intramolecular hydrogen bonds are formed. The inclusion of the flavonoid causes the γ-CD to lose its flexibility; inclusion of the flavonoid makes the complex less soluble than the pure cyclodextrin and precipitates out of the solution. This may facilitate, inter alia, complex formation, since the product is withdrawn from the reaction this way and, according to Le Chatelier, the equilibrium is more on the product side, but it also ensures lower solubility of the product.

It is also interesting that the peaks in both the β-CDs and the γ-CD are completely shifted. This speaks in favour of almost complete complex formation in solution, a point which is of particular interest for large-scale implementation.

Discussion of Preliminary Tests

The preliminary tests demonstrated that the complex formation with the cyclodextrins used takes place spontaneously and almost completely in solution. In addition, the exact conformation of the flavonoid in the CD cavity was determined as a function of the type of cyclodextrin. These points speak in favour of using cyclodextrins to increase the bioavailability and stability of the flavonoid taxifolin, but also of other flavonoids of the formula (I). In particular, the structure elucidation of the cyclodextrin/taxifolin complex permits interesting predictions to be made about the physicochemical behaviour of the complexes.

An increase in solubility can be expected for both β-CDs and γ-CDs. However, the increase in stability, which is achieved in particular through the encapsulation of ring B, is of particular interest. Since this was only the case with the β-CDs, the best results can be expected for this type of cyclodextrin. Moreover, the increase in permeability also speaks in favour of using the β-CDs. However, since both types of cyclodextrin can be used to improve physicochemical properties, the complex formation was carried out on a laboratory scale for both β-CD and γ-CD.

2. Production of Cyclodextrin Complexes

In order to use the complexes industrially, as well, the complexes have to be produced on a large scale. Various methods are available for this, but they have a significant influence on the solubility, encapsulation efficiency, and quality of the powder complex. In order to find the optimal method for producing a taxifolin/cyclodextrin complex, complexes with β- and γ-cyclodextrin were formulated and then analysed in greater detail. The methods can also be applied to all α, β, γ, and δ-cyclodextrins and their derivatives.

Materials: Freeze dryer (Martin Christ, Alpha 3-4 LSCbasic), spray dryer (BÜCHI Mini Spray Dryer B-290), homogenizer (IKA T 25 digital ULTRA-TURRAX® disperser), microwave (Siemens HF15M552), electronic stirrer (Variomag-USA based in Daytona Beach, Fla.)

β-cyclodextrin (CAVAMAX W7 FOOD from Wacker Chemie AG based in Munich, batch no.: 801153), γ-cyclodextrin (CAVAMAX W8 FOOD from Wacker Chemie AG based in Munich, batch no.: 801153), taxifolin (98.9% purity, Lavitol from Ametis JSC based in Amurskaja Oblast, Russia), water (dist.) acetone (ROTIPURAN®≥99.8%, p.a., Carl Roth), ethanol (ROTIPURAN®≥99.8%, p.a., Carl Roth), sodium hydroxide (≥98%, p.a., ISO, in pellets, Carl Roth), hydrochloric acid (1 mol/L 1 N standard solution, Carl Roth)

2.1. Beta Cyclodextrin

Since β-cyclodextrin was selected as a suitable cyclodextrin based on the preliminary tests, complexes were then produced using various methods and then examined based on their specific properties.

1:1 Physical Mixture

To produce the physical mixture as a reference substance, equimolar amounts of β-cyclodextrin and taxifolin were weighed out and mixed uniformly. The finished mixture was stored in a dry place at 25° C. room temperature, protected from light.

Solution 3-CD (SOLU β)

1000 mg taxifolin and 3730 mg beta-cyclodextrin were each weighed out in a 1:1 molar ratio and placed in two separate beakers. The beta-cyclodextrin was placed in a round bottom flask with ground joint together with 15 ml dist. water, put on a reflux condenser (Dimroth condenser), and the suspension was heated to 90° C. The taxifolin was then added to the cyclodextrin solution and the solution was refluxed for 5 minutes (600 rpm, 90° C.) until a clear solution formed.

The solution was then slowly brought to room temperature for 1 hour at 600 rpm and then cooled to 2° C. for 12 hours, the complex flocculating. The complex was separated off by vacuum filtration (0.45 μm membrane filter) and dried. After pulverization, the complex was hermetically sealed.

Kneading β-CD (KND β)

1000 mg taxifolin and 3730 mg beta-cyclodextrin were each weighed out in a 1:1 molar ratio and placed in a glass mortar. Next 2.4 ml dist. water was added to the beta-CD/taxifolin mixture. and the mixture was pestled continuously for 1 hour.

The complex was dried in a desiccator. After pulverization, the complex was hermetically sealed.

Slurry β-CD (SLUR β)

1000 mg taxifolin and 3730 mg beta-cyclodextrin were each weighed out a 1:1 molar ratio and placed in a shared beaker. 3.3 ml water was then added to the β-CD-taxifolin mixture. Next the solution was stirred for 24 h at 600 rpm and 25° C. with exclusion of oxygen, the complex forming in solution and flocculating. The complex was separated off by vacuum filtration (0.45 μm membrane filter) and dried. After pulverization, the complex was hermetically sealed.

Spray Drying 3-CD (SD β)

10,000 mg taxifolin and 37,300 mg β-cyclodextrin were each weighed out in a 1:1 molar ratio and placed in a shared beaker. 940 ml dist. water (25° C., 5% w/v) was then added to the β-CD-taxifolin mixture and the mixture was stirred for 30 minutes at 25° C. with a high-shear mixer (3000 min-1) until a concentrated suspension formed. This suspension was stirred for 24 h at 600 rpm and 25° C. with exclusion of oxygen in order to complete the complex formation. The solution was vacuum filtered (0.45 μm membrane filter) in order to remove undissolved flavonoid and cyclodextrin residues and the filtrate was then spray-dried.

Parameters: V=900 ml, T(in)=125° C.; pump rate: 20%; aspirator: 100%, spray gas: 55 mm; T(out)=71° C.

Freeze Drying β-CD (FD β)

1000 mg taxifolin and 3730 mg β-cyclodextrin were each weighed out in a 1:1 molar ratio and placed in a shared beaker. Next, 94 ml dist. water (5% w/v) was added to the β-CD-taxifolin mixture and stirred for 30 min at 30° C. with a homogenizer (3000 min-1) until a suspension formed. This suspension was stirred for 24 h at 600 rpm and 25° C. with exclusion of oxygen in order to complete the complex formation. The solution was vacuum filtered (0.45 μm membrane filter) in order to remove undissolved flavonoid and cyclodextrin residues and the filtrate was then cooled to −80° C. in centrifuge tubes for 24 hours and thus frozen. The tubes were then placed in the freeze dryer and the pressure was set to 0.05 mbar and the temperature to −30° C. The solution was freeze-dried in this way for 96 hours.

Yield:

An important point, especially for scaling up the process, is the yield of each of the methods. This allows the most efficient and thus the most cost-effective method to be selected.

Results:

| | | |
|---|---|---|
| Complex | Yield (mg) | Yield (%) |

| | | | |
|---|---|---|---|
| SOLU β | 4274 | mg | 90.3% |
| KND β | 4631 | mg | 97.9% |
| SLUR β | 3632 | mg | 76.8% |
| SD β | 27790 | mg | 58.75% |
| FD β | 4105 | mg | 86.7% |

Discussion: The yield was in particular very high in the kneading process (KND β), since losses only occur through residues on mortars, etc. In the case of SOLU β and SLUR β, part of the material dissolves in the solvent water and is separated out by filtration, which results in losses. By lowering the temperature, less complex goes into solution and the yield increases (SOLU β). In the case of FD β and especially SD β, the comparatively low yields can be explained by the fact that these processes were carried out on a laboratory scale. In this case, the yields are low, e.g. due to attachment to the spray tower (SD β) or loss of material during venting (FD β). In addition, undissolved flavonoid and cyclodextrin residues were removed before drying. These problems can be eliminated in a large-scale implementation, however.

Active Ingredient Content:

The active ingredient content is an important parameter that can vary with different methods. One reason for this is the different water content and possible degradation during the manufacturing process.

100 mg of the samples was weighed out and completely dissolved in 50 ml dist. water. The stock solution was then diluted by a factor of 100 and transferred to a vial. The taxifolin concentration was then determined by HPLC and thus the taxifolin content of the complex was calculated.

| | Weighed | Complex | Taxifolin | Taxifolin |
|---|---|---|---|---|
| | in | concentration | concentration | content in |
| Complex | (mg) | (mg/mL) | (mg/mL) | complex |
| SOLU β | 101.66 | 0.20 | 0.0396 | 19.8% |
| KND β | 100.17 | 0.20 | 0.0398 | 19.9% |
| SLUR β | 100.60 | 0.20 | 0.378 | 18.9% |
| SD β | 100.90 | 0.20 | 0.418 | 20.9% |
| FD β | 100.63 | 0.20 | 0.346 | 17.3% |

The theoretical target value for the taxifolin concentration is 21.1%, with SD β being the closest to this value. All other complexes are around 20%, with the exception of FD β, which contains only 17.3% taxifolin. The low taxifolin content of the freeze-dried complex is probably due to the preparation, wherein undissolved taxifolin residues were filtered off. All of the complexes contain sufficient taxifolin for the formulation of various pharmaceutical forms of administration.

Various measurement methods are available for quantitatively determining the efficiency of the encapsulation method. One very popular method is dynamic differential calorimetry (DSC), with which the residual content of the free active ingredient can be determined using characteristic endothermic peaks (approx. 240° C. for taxifolin). Since the active ingredient-cyclodextrin complex has a different decomposition point or melting point, a high degree of encapsulation efficiency can be concluded indirectly from the lack of the “active ingredient peak”.

It is therefore particularly interesting to compare the sample peaks to the peaks of the pure active ingredient, the pure cyclodextrin, and an equimolar, physical mixture (active ingredient: cyclodextrin). The latter serves as a reference for the samples, since in a physical mixture the active ingredient is in the free, uncomplexed form (encapsulation efficiency=0%). A complete absence of the active ingredient peak at 240° C. corresponds to an encapsulation efficiency of 100%. The individual samples can be compared to one another and to the physical mixture based on the surface area of the characteristic active ingredient peak of said individual samples. The main advantage of this measuring method is, on the one hand, the very high precision, and, above all, the possibility of measuring the samples in the solid state. This prevents the complex equilibrium from being influenced or readjusted by water or other solvents.

A characteristic active ingredient peak can no longer be found in the samples SOLU β, SD β, and FD β. In addition, the intensity of the broad endothermic peak between 70° C. and 100° C. clearly decreases compared to the reference samples. This indicates that less water escapes from the cyclodextrin cavity during heating, as the latter is occupied by the flavonoid. The DSC thermograms therefore show that in these samples the flavonoid is present entirely as a β-CD complex and the encapsulation efficiency is 100%.

In the samples KND β and SLUR β, on the other hand, characteristic active ingredient peaks can still be seen, but the intensity decreases compared to the physical mixture, which indicates a complex formation. The intensity of the endothermic peak in the range between 70° C. and 100° C. can be roughly compared to the intensity of the physical mixture. Both points indicate that encapsulation in the CD cavity has taken place, but is incomplete. Based on the surface areas, efficiencies of 12.02% (KND β) or 12.98% (SLUR β) can be calculated. It is noticeable here that in the methods with complete encapsulation, both flavonoid and cyclodextrin were completely in solution, at least at one point in time. This is not the case with KND β and SLUR β, where there were only suspensions. Since the complex formation basically only takes place in solution, the equilibrium, which is strongly on the side of the complex, is established very quickly if both starting materials are present in dissolved form.

In the kneading or suspension process, complex formation also only takes place in solution, which is why significantly longer reaction times and/or higher temperatures are necessary in these processes. In this case, the parameters were not chosen optimally, which is why complete encapsulation was not achieved, but complex formation is possible using these methods.

FITR Analyses

FT-IR spectroscopy is used to analyse the molecular interactions between the functional groups of the flavonoid and the cyclodextrin. This should make it possible to draw conclusions about the spatial structure of the taxifolin/β-CD complex and confirm complex formation.

The FT-IR spectra show quite significant differences between SOLU β, SD β, FD β and KND β or SLUR β. In principle, all characteristic cyclodextrin peaks can also be found in the complexes, with the exception of SOLU β. There were differences in particular with the taxifolin peaks, which are shifted or disappear completely when complexes are formed.

Saturation Solubility in Dist. Water (HPLC)

The last most important point in terms of comparing the production methods to one another is solubility in distilled water. The solubility of the complex has a direct influence on bioavailability, because only dissolved complexes/active ingredients can pass the epithelial cells of the GI tract. Moreover, the samples were examined for rel. substances in order to identify possible degradation of the active ingredient during the production process.

Method:

Reference Measurement (Taxifolin)

10 mg taxifolin (Lavitol® 98.9% purity) was placed in a vial with 5 ml dist. water to make a saturated solution and shaken for 60 minutes. The solution was then transferred to a vial using a syringe with an HPLC filter (0.22 μm) and then measured undiluted (HPLC DAD-254 nm).

Sample Measurement

500 mg of the sample was placed in a vial with 6 ml dist. water to make a saturated solution and shaken for 60 minutes. The solution was then transferred to a vial using a syringe with an HPLC filter (0.22 μm), diluted 10:1 with distilled water to prevent oversaturation, and then measured (HPLC DAD-254 nm). The taxifolin concentration was calculated in mg/ml based on the peak area, taking into account the dilution.

Results:

| | Surface | Solubility of | Rel. substances |
|---|---|---|---|
| Name | area | taxifolin in mg/ml | peak |
| Taxifolin reference | 12062743 | 0.7496 | 3430343 |
| 1:1 phys. mixture | 34442051 | 22.3834 | — |
| SOLU β | 32145223 | 20.8555 | — |
| KND β | 31966583 | 20.7366 | — |
| SLUR β | 23599460 | 15.1707 | — |
| SD β | 38177574 | 24.8682 | — |
| FD β | 36450181 | 23.7192 | — |

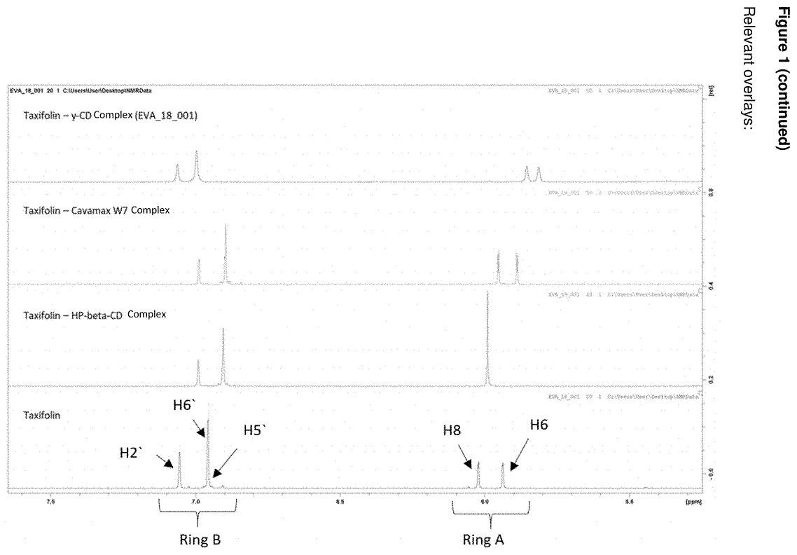

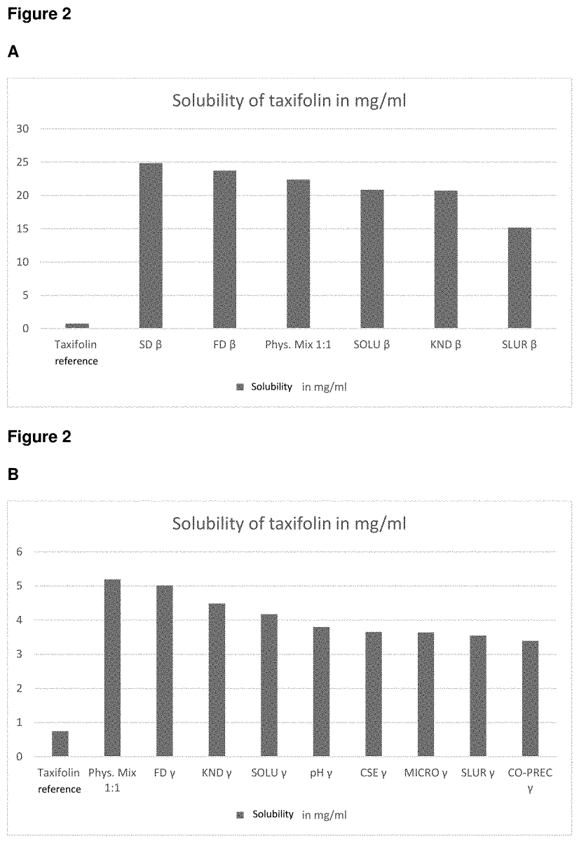

The results of the solubility tests are also illustrated graphically in FIG. 2A.

Inclusion complexes with β-CD massively increase the saturation solubility of the flavonoid taxifolin. This effect is particularly pronounced for the formulations SD β and FD β. However, KND β, SOLU β, and SLUR β were also able to greatly increase the saturation concentration, although this effect was less pronounced with SLUR β.

The physical 1:1 mixture also achieved very good results, which is due to the formation of complexes in solution. The physical mixture actually represents the maximum possible upper limit for improving solubility, since the complex can form under maximum saturation, i.e., optimal conditions.

Nevertheless, the taxifolin concentration of the formulations SD β and FD β exceeds this value; this is probably due to oversaturation of the solution due to the small particle size and thus the large surface area of the material.

SOLU β and KND β do not form a supersaturated solution due to their particle size and are therefore just below the maximum value of the phys. mixture. However, since KND β has only very low encapsulation efficiency, it is possible that the improvement in solubility occurs through complex formation in solution, similar to the phys. mixture. The improvement in solubility is lowest with the SLUR βformulation; higher-level complexes may form during the production process.

In conclusion, it may be said that β-CD is ideally suited for formulating water-soluble, bioavailable inclusion complexes with taxifolin and similar flavonoids. Moreover, these complexes are also suitable for formulating bioavailable pharmaceutical dosage forms.

2.2 γ-cyclodextrin

Since γ-cyclodextrin was selected as a suitable cyclodextrin based on the preliminary tests, complexes were then produced using various methods and then examined based on their specific properties.

1:1 Physical Mixture

To produce the physical mixture as a reference substance, equimolar amounts of γ-cyclodextrin and taxifolin were weighed out and mixed evenly. The prepared mixture was protected from light and stored in a dry place at 25° C. room temperature.

Slurry γ-CD (SLUR γ)

1000 mg taxifolin and 4266 mg γ-cyclodextrin were each weighed out in a 1:1 molar ratio and placed in separate beakers. Then 40 ml dist. water was added to the γ-CD to obtain a concentrated, clear solution (γ-CD 11.5% w/v). Next, the flavonoid was added to the solution. This solution was then stirred for 12 hours at 25° C. and 600 rpm.

The flocculated complex was vacuum filtered (0.45 μm membrane filter) and dried in a desiccator. After pulverization, the complex was stored airtight and protected from light.

Solution γ-CD (SOLU γ)

1000 mg taxifolin and 4266 mg γ-cyclodextrin were each weighed out in a 1:1 molar ratio and placed in separate beakers. Then 80 ml dist. water was added to the γ-CD and heated to 50° C. Next, the flavonoid was added to the solution. This solution was then stirred for 1 hour at 50° C. and 600 rpm until a clear solution formed. The complex was then cooled to 2° C. for 12 hours, so that the complex flocculated. The flocculated complex was vacuum filtered (0.45 μm membrane filter) and dried in a desiccator. After pulverization, the complex was stored airtight and protected from light.

Co-Precipitation γ-CD (CO-PREC γ)

1000 mg taxifolin and 4266 mg γ-cyclodextrin were each weighed out in a 1:1 molar ratio and placed in separate beakers. Next, 40 ml dist. water was added to the γ-CD to obtain a concentrated, clear solution (γ-CD 11.5% w/w). This was heated to 50° C. The flavonoid was completely dissolved in 10 ml acetone and added to the solution. Next this solution was stirred for 3 hours at 50° C. and 600 rpm and then cooled to room temperature for 12 hours. The flocculated complex was vacuum filtered (0.45 μm membrane filter) and dried in a desiccator. After pulverization, the complex was stored airtight and protected from light.

H₂O γ-CD kneading (KND γ)

1000 mg taxifolin and 4266 mg γ-cyclodextrin were each weighed out in a 1:1 molar ratio and placed in a glass mortar. Next, a total of 18 ml distilled water was added to the γ-CD/taxifolin mixture and pestled continuously for 1 hour.

The complex was dried in a desiccator. After pulverization, the complex was stored airtight and protected from light.

γ-CD Common Solvent Evaporation (CSE γ)

1000 mg taxifolin and 4266 mg γ-cyclodextrin were each weighed out in a 1:1 molar ratio and placed in a beaker. Then 25 ml dist. water was heated to 60° C. at 600 rpm and the taxifolin/CD mixture was added and stirred until a clear solution formed. Next the solution was stirred at 600 rpm until all of the water had evaporated. The complex was dried in a desiccator. After pulverization, the complex was stored airtight and protected from light.

γ-CD Microwave Irradiation (MICRO γ)

1000 mg taxifolin and 4266 mg γ-cyclodextrin were each weighed out in a 1:1 molar ratio and placed in separate beakers. Next, 40 ml dist. water was added to the γ-CD to obtain a concentrated, clear solution (γ-CD 11.5% w/w). This was stirred at 25° C., 600 rpm for 5 minutes and the flavonoid was added to the solution. This solution was then stirred for 30 minutes at 600 rpm and then heated in a microwave to 70° C. at 90 Watts for 2 minutes. The solution became completely clear. The complex was then stirred for 3 hours at 600 rpm and cooled to room temperature for 12 hours. The flocculated complex was vacuum filtered (0.45 μm membrane filter) and dried in a desiccator. After pulverization, the complex was stored airtight and protected from light.

γ-CD pH Shift (pH γ)

1000 mg taxifolin and 4266 mg γ-cyclodextrin were each weighed out in a 1:1 molar ratio and placed in separate beakers. Next, 24 ml of a NaOH solution (0.18 mol/L) was added to the γ-cyclodextrin and the mixture was stirred at 600 rpm and room temperature. The flavonoid was then added and the mixture was stirred for 1:30 min at 600 rpm and 25° C. until a clear solution had formed. The solution was then adjusted to pH 2 with HCl (1 mol/L), the solution immediately becoming cloudy. The suspension was then stirred at 600 rpm and 25° C. for a further 1.5 hours. The precipitate was vacuum filtered (0.45 μm membrane filter) and dried in a desiccator. After pulverization, the complex was stored airtight and protected from light.

γ-CD Freeze Drying (FD-γ)

1000 mg taxifolin and 4266 mg γ-cyclodextrin were each weighed out in a 1:1 molar ratio and placed in a shared beaker. Next, correspondingly 265 ml dist. water (2.5% w/v, 25° C.) was added to the γ-CD-taxifolin mixture and the mixture was stirred for 30 min with a homogenizer (3000 min-1) until a clear solution was formed. The solution was vacuum filtered (0.45 μm membrane filter) in order to remove undissolved flavonoid and cyclodextrin residues and the filtrate was then cooled to −80° C. in centrifuge tubes for 24 hours and thus frozen. The tubes were then placed in the freeze dryer and the pressure was set to 0.05 mbar and the temperature to −30° C. The solution was freeze-dried in this way for 96 hours.

Specification of the γ-CD Complexes

The samples were examined using different analytical methods in order to be able to differentiate between the complexing methods and the complexes produced thereby. On the one hand, emphasis was placed on a quantitative analysis (yield, DSC, solubility) and also on qualitative analysis by FTIR.

Yield: An important point, especially for scaling up the process, is the yield of each of the methods. This allows the most efficient and thus the most cost-effective method to be selected.

Results:

| Complex | Yield (mg) | Yield (%) |
|---|---|---|
| SLUR γ | 3947 mg | 75.0% |
| SOLU γ | 4293 mg | 81.5% |
| CO-PREC γ | 4434 mg | 84.2% |
| KND γ | 4970 mg | 94.4% |
| CSE γ | 3568 mg | 67.8% |
| MICRO γ | 2758 mg | 52.3% |
| pH γ | 2391 mg | 45.4% |
| FD γ | 4908 mg | 93.2% |

Discussion: In general, the kneading method provides a high yield; losses are only caused by residues on the device used (mortar, bowl, etc.). In all methods in which the complex was precipitated from the solution (SLUR, SOLU, CO-PREC, MICRO, pH), low yields can be explained by the fact that the complex also largely dissolves in the distilled water, but precipitates out. This has only a minor effect if the water has been cooled down significantly (SOLU), but it has a major effect if it is filtered immediately after only a short reaction and precipitation time (pH, MICRO).

Very high yields are possible with spray drying, but experience has shown that the yields in the model experiment are low, since with a small amount of powder a relatively large portion remains on the walls in the spray tower.

Very high yields are possible with freeze drying, since no complex powder remains behind or deposits apart from residues in the vessel and losses during repackaging.

Active Ingredient Content (HPLC)

The active ingredient content is an important parameter that can vary with different methods. One reason for this is the different water content and possible degradation during the manufacturing process.

Method: 100 mg of the samples was weighed out and completely dissolved in 50 ml dist. water. The stock solution was then diluted by a factor of 100 and transferred to a vial. The taxifolin concentration was then determined by HPLC and thus the taxifolin content of the complex was calculated.

| | Weighed | Complex | Concentration | Complex |
|---|---|---|---|---|
| | in | concentration | of taxifolin | content |
| Complex | (mg) | (mg/mL) | (mg/mL) | of taxifolin |
| SLUR γ | 100.43 | 0.20 | 0.0312 | 15.6% |
| SOLU γ | 101.29 | 0.20 | 0.0336 | 16.8% |
| CO-PREC γ | 100.25 | 0.20 | 0.0306 | 15.3% |
| KND γ | 102.90 | 0.21 | 0.03486 | 16.6% |
| CSE γ | 101.31 | 0.20 | 0.0326 | 16.3% |
| MICRO γ | 102.96 | 0.21 | 0.03255 | 15.5% |
| pH γ | 102.04 | 0.20 | 0.0326 | 16.3% |
| FD γ | 102.08 | 0.20 | 0.0360 | 18.0% |

Discussion: The theoretical target value for the taxifolin content for the γ-cyclodextrin complexes is 19%. FD γ comes closest to this; all of the other complexes have a similar active ingredient content between 15.5% and 17%.

All of the complexes contain sufficient taxifolin for the formulation of certain pharmaceutical dosage forms, although administration in capsule/tablet form is more difficult due to the correspondingly high dosage.

DSC Analyses

Various measurement methods are available for quantitatively determining the efficiency of the encapsulation method. One very popular method is dynamic differential calorimetry (DSC), with which the residual content of the free active ingredient can be determined using characteristic endothermic peaks (approx. 240° C. for taxifolin). Since the active ingredient-cyclodextrin complex has a different decomposition point or melting point, a high degree of encapsulation efficiency can be concluded indirectly from the lack of the “active ingredient peak”.

It is therefore particularly interesting to compare the sample peaks to the peaks of the pure active ingredient, the pure cyclodextrin, and an equimolar, physical mixture (active ingredient: cyclodextrin). The latter serves as a reference for the samples, since in a physical mixture the active ingredient is in the free, uncomplexed form (encapsulation efficiency=0%). A complete absence of the active ingredient peak at 240° C. corresponds to an encapsulation efficiency of 100%. The individual samples can be compared to one another and to the physical mixture based on the surface area of the characteristic active ingredient peak of said individual samples. The main advantage of this measuring method is, on the one hand, the very high precision, and, above all, the possibility of measuring the samples in the solid state. This prevents the complex equilibrium from being influenced or readjusted by water or other solvents.

Discussion: Broad endothermic peaks in the range between 70° C. and 100° C. indicate the escape of residual water during heating. Otherwise the thermograms of the γ-CD complexes differed quite fundamentally from the thermograms of the β-CD complexes. For example, except for pH γ, all of the complex samples no longer have a characteristic active ingredient peak that corresponds to the physical mixture. This indicates complete encapsulation, as free flavonoid can no longer be detected. However, these samples show peaks in the range of 245° C.-250° C., the surface area of which in some cases significantly exceeds that of the physical mixture. These peaks could indicate the decomposition of the γ-CD/Taxifolin complex or the supramolecular complex agglomerates. These agglomerates are typical of γ-CD complexes and are often described in the literature.

With the pH shift method, however, free, uncomplexed flavonoid can still be detected, which indicates incomplete encapsulation. This could be due to the short reaction time with this procedure, since the flavonoid can only be encapsulated in the CD cavity in an uncharged, neutral state. The reaction between flavonoid and cyclodextrin therefore only takes place in the short time window between protonation of the flavonoid and precipitation of the complex, which can lead to incomplete encapsulation.

Otherwise complete encapsulation was achieved by all methods used, which is due to the very high complex stability constant KS for γ-CD/taxifolin complexes. Large-scale production is greatly simplified by this, but complex stability that is too high could also have a retarding effect on the flavonoid release, and the formation of supramolecular agglomerates can lead to a “spring-parachute effect”, wherein the complex again precipitates out of solution after dissolution.

FITR Analyses

FT-IR spectroscopy is used to analyse the molecular interactions between the functional groups of the flavonoid and the cyclodextrin. This should make it possible to draw conclusions about the spatial structure of the taxifolin/γ-CD complex and confirm the complex formation.

The reference spectra are as expected and correspond to the literature. The spectrum of the cyclodextrin also shows all characteristic peaks, comparable to those of the β-cyclodextrin. The physical mixture shows only superimposed spectra of the cyclodextrin and the flavonoid.

All characteristic cyclodextrin peaks can be found in the complex spectra; in fact the spectra of the complexes and of γ-cyclodextrin are almost identical. Compared to the physical mixture, there were differences in particular in the taxifolin peaks, which are shifted or disappear completely due to complex formation. This is where the complexes differ significantly from the physical mixture. This indicates a hindrance in the oscillation of these functional groups due to complex formation/interaction with the cyclodextrin.

All of the complex samples showed almost identical spectra, which indicates equivalence of the methods and can be explained by the high complex stability of the taxifolin/γ-cyclodextrin complex. The spectra indicate the formation of a real, typical inclusion complex between taxifolin and γ-cyclodextrin.

Saturation Solubility in Dist. Water (HPLC)

The last most important point in terms of comparing the production methods to one another is solubility in dist. water. The solubility of the complex has a direct influence on bioavailability, because only dissolved complexes/active ingredients can pass the epithelial cells of the GI tract. Moreover, the samples were examined for rel. substances in order to identify possible degradation of the active ingredient during the production process.

Method

Reference Measurement (Taxifolin)

10 mg taxifolin (Lavitol® 98.9% purity) was placed in a vial with 5 ml dist. water to make a saturated solution and shaken for 60 minutes. The solution was then transferred to a vial by means of a syringe with an HPLC filter (0.22 μm) and then measured (HPLC DAD-254 nm).

Sample Measurement

300 mg of the sample was placed in a vial with 5 ml dist. water to produce a saturated solution and shaken for 60 minutes. The solution was then transferred to a vial using a syringe with an HPLC filter (0.22 μm) and then measured undiluted (HPLC DAD-254 nm).

Results

| | Surface | Solubility | Rel. substances |
|---|---|---|---|
| Name | area | in mg/ml | peak |
| Taxifolin | 12062743 | 0.7496 | 3430343 |
| reference | | | |
| Phys. mix 1:1 | 78880276 | 5.1944 | — |
| SLUR γ | 54175858 | 3.5511 | — |
| SOLU γ | 63580320 | 4.1767 | — |
| CO-PREC γ | 51836311 | 3.3954 | — |
| KND γ | 68286523 | 4.4897 | — |
| CSE γ | 55704140 | 3.6527 | — |
| MICRO γ | 55497443 | 3.6390 | — |
| pH γ | 57881093 | 3.7975 | — |
| FD γ | 76185885 | 5.0152 | — |

The results of the solubility studies are illustrated in FIG. 2B.

Discussion: The optimum production method can be selected based on saturation solubility. Freeze-drying and spray-drying and the kneading process appear to be particularly effective. This allowed complexes with maximum saturation solubility to be achieved. This could be due to the small particle size of the freeze-dried complexes, on the one hand, but also, on the other hand, to more complete complex formation. In addition, the formation of agglomerates and the arrangement in higher-level structures is an important factor.

It is also interesting that the physical mixture achieves maximum saturation solubility, that is, 5.194 mg/ml is the maximum solubility of taxifolin that can be achieved with γ-CD. On the one hand, this indicates that the reaction equilibrium is reached after stirring for 1 hour; on the other hand, it indicates that the reaction in solution is also strongly on the side of the complex and maximum solubility can be achieved due to the lack of agglomerate formation. In addition, since the solubility of the freeze-dried and spray-dried complexes and the kneaded complex is very close to the solubility of the physical mixture, it can be assumed that the solubility of these complexes is almost maximal. However, the saturation solubilities of the γ-CD complexes are significantly lower than those of the β-CD complexes.

Discussion of CD Complex Production

Due to higher complex solubility, the increase in permeability, and the protection of the catechol group, β-CD should be clearly preferred to γ-CD. In addition, γ-CD complexes have a greater tendency to form agglomerates and to delay the release of active ingredients.

Freeze drying and spray drying are particularly suitable as methods, since real inclusion complexes with very high encapsulation efficiency are formed when these methods are used. This is reflected in the high saturation solubility and the good dissolution behaviour of the formulations.

Spray drying is particularly interesting for the production of orally ingestible formulations, since the production costs are comparatively low compared to freeze drying for a comparable product.

Freeze drying is particularly suitable for the production of intravenous preparations, whereby special derivatives of β-CD (e.g. hydroxypropyl-β-CD or sulfobutyl ether-β-CD) are used due to their better water solubility and lower toxicity.

Kneading is also an attractive method, as the manufacturing costs are very low. In addition, this method can be implemented on an industrial scale without any problems (e.g., in a high-shear wet granulator, an Eirich mixer, or an industrial kneader), wherein high throughput rates with a short processing time are possible. However, the disadvantage of this method is the very low encapsulation efficiency.

Other production methods are also fundamentally suitable for producing complexes, especially on a laboratory scale. However, large-scale production using these methods is significantly more cost-intensive. In addition, for some methods, either large water tanks (SOLU, SLUR, CSE) or special devices (pH, MICRO) are required, and the yields are also quite low.

Agglomerates as a Limiting Factor for Solubility

An important point to be observed, especially with natural cyclodextrins and especially with γ-cyclodextrins, is possible agglomerate formation of the complexes. This problem has already been identified for ubiquinone/γ-CD complexes and had an enormous influence on the solubility of the product. The complexes are arranged in a solid crystal structure to form supramolecular complexes. This massively reduces the surface area and also the hydration of the individual complexes. Even with theoretically high solubility of the complexes, a cloudy, characteristically opalescent suspension is formed.

The driving force for the agglomerate formation is a negative enthalpy, because the complexes form highly ordered, crystal-like structures and thus have a stable and low-energy conformation. However, when the supramolecular complexes are formed, the order of the system is increased, the entropy decreases, and ΔS0 becomes positive. According to the Gibbs-Helmholtz equation it follows that the formation of these supramolecular complexes decreases as temperature increases.

ΔG₀=ΔH₀−T×ΔS₀<0

This was confirmed in a number of test series, because an opalescent complex suspension (SLUR γ) could be completely clarified by heating from 20° C. to 50° C. with the same amount of water. However, this alone is not yet an indication of agglomerate formation, since this effect could also have come about through increased solubility of the complex in warmer water.

Experiments with chaotropic substances were carried out in order to be able to prove the limitation of solubility due to agglomerate formation. These substances hinder the formation of hydrogen bonds, which stabilize the complexes in the highly ordered structure. At the same time, the highly ordered structure of the solvent, water, is broken and hydrophobic effects are thus reduced.

Specifically, another opalescent suspension was prepared (250 mg SLUR γ complex powder in 20 ml dist. water) and then 10 g urea was added thereto. The suspension cleared completely after stirring for 10 min at 600 rpm without the temperature being increased. The solubility could be significantly increased by breaking up the aggregates.

This can also explain the comparatively high solubility of the SD, FD, and KND complexes. Because the fast drying process (SD, FD) or the high shear forces (KND) prevent an arrangement in highly ordered complexes. Therefore, complexes of these three methods also have the highest saturation solubility and the best dissolution behaviour. The addition of a hydrophilic polymer (e.g. PEG 6000) can also prevent these higher-level structures from developing. In all of the other manufacturing methods, an arrangement in the supramolecular complex is made possible and even promoted to some extent by the long precipitation time and temperature reduction. For this reason, freeze-drying, spray-drying, and kneading are the most interesting methods of complex formation for reducing supramolecular complexes.

Ternary Complexes

A screening was carried out in order to investigate which water-soluble polymers are particularly suitable for improving the stability and dissolving power of flavonoid-cyclodextrin complexes. First a saturated taxifolin-CD complex solution was prepared and then various water-soluble polymers were added (0.25% w/v). The solution was left to stand for 96 hours and the recrystallization was then compared to the polymer-free solution.

| Polymer used | Recrystallization | |
|---|---|---|
| (0.25% w/v) | after 96 hours | Comment |
| No polymer (reference) | Pronounced | |
| PVP K30 | Very strongly pronounced | Worsening |
| PVP/VA | Very strongly pronounced | Worsening |
| HPMC | Strongly pronounced | Worsening |
| MC | Pronounced | No change |
| Carbomer | Pronounced | No change |
| Poloxamer 188 | None | Improvement |
| PEG 6000 | None | Improvement |
| PVA | Hardly any | Improvement |
| PEG/PVA (Kollicoat ® | None | Improvement |
| IR) | | |
| Xanthan gum | Pronounced | No change |
| Gellan | Pronounced | No change |
| Eudragit E100 | Strongly pronounced | Solution in 0.1N |
| | | HCl, worsening |
| Chitosan | Pronounced | Solution in 0.1N |
| | | HCl, no change |
| Pectin | Pronounced | No change |
| Na-CMC | Strongly pronounced | Worsening |
| Alginic acid | Pronounced | No change |
| Collagen hydrolysate | Pronounced | No change |
| Maltodextrin | Pronounced | No change |

The results clearly demonstrate that polymers with distinctive hydrogen-bond acceptors (PVP, PVP/VA, Eudragit E100, and cellulose derivatives) lead to worsening due to strong interaction with the active ingredient. The polymer-active ingredient complex precipitates and Ks drops. In addition, there is no interaction with the typical biopolymers, neither with the active ingredient nor with the cyclodextrin, so that the dissolution behaviour of the active ingredient is not changed.

In contrast, PEG 6000, Kollicoat IR, and Poloxamer 188 are of particular interest. These polymers are built up from ethylene oxide blocks and show very promising properties. The interaction with the hydroxyl groups of the flavonoid is not so strong that precipitation occurs; at the same time, the polymers also interact with the hydroxyl groups of the cyclodextrin. This increases complex stability. The same can be seen with polyvinyl alcohol (PVA). The interaction of the hydroxyl groups of the polymer with the flavonoid and the cyclodextrin is less pronounced than with the ethylene oxide polymers, however.

This demonstrated that the use of water-soluble polymers increases complex stability and improves dissolution behaviour.

For this purpose, it is sufficient to physically mix the water-soluble polymer and the finished flavonoid/CD complex, since a ternary complex forms after dissolution in solution. The integration of the polymer can also take place before or during the complex formation, however. For example, small amounts of the polymer can be added to the solution prior to spray drying or freeze drying. Moreover, small amounts of the polymer can also be added to the solution which is used to moisten the Taxifolin/β-CD paste. Concentrations between 0.0025%-2% w/v in the final solution would be reasonable; most often around 0.25% w/v is used.

3. Preparation of a Solid Dispersion

In contrast to other active ingredients such as β-carotene which are poorly soluble in water due to their lipophilicity, taxifolin has a very hydrophilic structure. It is precisely the many hydroxyl groups and the keto group at position 4 that allow hydrogen bonds and should, in theory, ensure good water solubility. However, similar to itraconazole, the crystalline structure prevents an efficient solution. For this reason, solid dispersions with various polymers are mainly used for this group of active ingredients.

The active ingredient is distributed in a molecularly dispersed manner in the polymer, thereby dissolving the crystalline structure. If the polymer:active ingredient dispersion is now added to water, the energetically stable crystalline structure does not have to be broken up first, but instead the active ingredient can be dissolved immediately as long as it is polar enough. The prerequisite for this is relatively strong molecular bonds between the active ingredient and the polymer and thus crystallization-inhibiting effect for the polymer. This is the only way to prevent the active ingredient from recrystallising again and thus becoming insoluble.

3.1 Preliminary Tests

In order to find the optimal polymer, solid dispersions were formulated with typical pharmaceutical polymers as well as various biopolymers. PVP, PEG, PVA/VA, Soluplus® (polyvinyl caprolactam-polyvinyl acetate-polyethylene glycol copolymer), Carbomer (polyacrylic acid), PVA (polyvinyl alcohol), Eugragit E, HPMC (hydroxypropylmethyl cellulose), HPC (hydroxypropyl cellulose), MC (methyl cellulose), Na-CMC (sodium carboxymethyl cellulose), maltodextrin, shellac, collagen hydrolysate, chitosan, gellan, xanthan and alginic acid were tested.

The solvent evaporation method (CSE) was used to prepare the solid dispersions. In this method, both the polymer and the active ingredient (taxifolin) are dissolved in the same solvent and this is then evaporated. The taxifolin is optimally stabilized in its amorphous form, so that water solubility and dissolution rate can be increased drastically.

The polymer and taxifolin were dissolved in the solvent in various ratios (1:1-12:1 w/w) and then dried in a dark, well-ventilated location.

In order to identify possible recrystallization, a few drops of the polymer flavonoid solution were placed on a cover slip and, after drying, examined for taxifolin crystals under a light microscope (film casting).

Results: In the case of solid dispersions with biopolymers, the taxifolin:polymer interaction was too low and the taxifolin flocculated again. This did not result in any improvement in solubility in water.

In the case of the pharmaceutical polymers, especially in the case of polymers with carbonyl groups, a strong interaction was found, so that the taxifolin was effectively prevented from recrystallizing. However, if the polymer:taxifolin ratio was too low (sometimes anything below 9:1), the dispersions flocculated and thus became water-insoluble.

Only the solid dispersions with Eudragit® E showed no signs of recrystallization, even at low polymer:taxifolin ratios, and increased solubility in water and the dissolution rate of taxifolin significantly from an Eudragit® E:taxfolin ratio of 1:1 w/w, without the solid dispersion flocculating.

Discussion: Biopolymers, which are mostly sugar derivatives, are unsuitable carriers for a solid dispersion with taxifolin. This can be explained by the numerous hydroxyl and ether groups and the lack of carbonyl groups, since polyphenols interact much more strongly with the latter. This is shown particularly well in the fact that taxifolin is very soluble in acetone and ethyl acetate, while it is insoluble in diethyl ether. Moreover, most biopolymers are insoluble in organic solvents and too heat-labile for hot-melt extrusion (HME), which makes large-scale production difficult.

Water-soluble synthetic polymers that are sufficiently soluble in organic solvents and approved for human consumption are therefore of particular interest. In theory, polyvinylpyrollidone and its derivatives are particularly suitable for this purpose, since these polymers are water-soluble and have even been approved as additives in food. In addition, the pyrollidone ring forms strong hydrogen bonds with the phenolic groups of the flavonoid, so that the taxifolin is stabilized in its amorphous form and does not recrystallize.

The only problem with PVP as a carrier matrix is that the bonds between the flavonoid and the polymer are too strong. Especially with low polymer:active ingredient ratios, this can mean that the polymer cannot form hydrogen bonds with water, since the flavonoid displaces the water. If this happens, neither the polymer nor the flavonoid can be dissolved and the dispersion flocculates.

This happened with all dispersions with a PVP:taxifolin ratio of less than 6:1 and only starting at 9:1 did significant improvements in solubility become apparent. The increase in solubility was enormous (factor 42), but the low drug loading of only 10% makes it difficult to use. This is not a problem with potent active ingredients, but if a 500 mg single dose of taxifolin were assumed, an additional 4500 mg PVP would have to be taken. This would correspond to an amount of about 5-10 tablets and, in addition, the ingestion of such large amounts of a synthetic polymer is disadvantageous.

The experiments with polyvinylpyrollidone co-polymers such as Kollidon® VA 64, a co-polymer made from PVP and vinyl acetate, are also of interest. The carbonyl groups of the ester bonds (vinyl acetate) and the pyrollidone ring (PVP) interact strongly with the flavonoid, which can effectively prevent recrystallization. However, similar to PVP, this strong interaction makes interaction with water molecules difficult, making the dispersion insoluble in water. Since the vinyl acetate group also interacts more poorly with water molecules than the pyrollidone ring, the drug loading must be reduced to below 10%, which makes the polymer unsuitable for this use.

Eudragit®E, on the other hand, is ideally suited as a carrier for solid dispersions with taxifolin or similar flavonoids. This is due to the intermolecular forces between the carbonyl group of the methacrylic ester and the hydroxyl groups of the flavonoid, similar to PVP. This stabilizes the flavonoid in its amorphous form, which considerably improves its solubility in water. The difference with respect to PVP is that the cationic aminoalkyl groups of the Eudragit make the polymer water-soluble, even if it interacts strongly with the flavonoid.

In principle, various manufacturing processes are available for large-scale production, with spray drying (SD) and hot melt extrusion (HME) being the most suitable methods.

3.2 Film Casting

A polymer screening with subsequent film casting is often carried out in order to find the most suitable polymer or polymer:active ingredient ratio as efficiently as possible. Here, the active ingredient and the polymer are dissolved in different proportions in an organic solvent and the solution is then placed on a glass cover slip. After drying, the sample is examined under a light microscope for recrystallization of the active ingredient. If no crystals can be found, the polymer or polymer:active ingredient ratio is suitable for producing a solid dispersion

Method: Taxifolin and Eudragit® E100 were each dissolved in ethanol in 1:1, 1:2, and 1:3 ratios and then placed on a cover slip. After drying, the cover slips were examined for taxifolin crystals under a light microscope.

Results: Recrystallization could not be found for any of the cover slips. The solid dispersion was glass-like and significantly darker than dissolved taxifolin or Eudragit® E100 alone.

Discussion: In general, basic polymethacrylates are ideal for producing solid dispersions with taxifolin and flavonoids. On the one hand, this is due to the very strong flavonoid-polymer interaction, with hydrogen bonds between the carboyl groups of the polymer and the phenolic hydroxyl groups of the flavonoid and ionic bonds especially playing a role. On the other hand, the solid dispersion does not flocculate in acidic solution (compared to solid dispersions with other polymers), since the polymer is cationically charged and thus becomes extremely water-soluble.

3.3 Production of Solid Dispersions with Eudraqit® E

Materials: Electronic stirrer (Variomag-USA based in Daytona Beach, Fla.) Taxifolin (98.9% purity, Lavitol from Ametis JSC based in Amurskaja Oblast, Russia), ethanol (ROTIPURAN®≥99.8%, p.a., Carl Roth), basic polymethacrylate Eudragit® E100 (Evonik Industries, Essen)

Production Methods

Common Solvent Evaporation 1:1 (CSE 1:1)

1000 mg of Eudragit® E100 was weighed out and dissolved in 25 ml ethanol. 1000 mg taxifolin was then weighed out and dissolved in 15 ml ethanol. The two solutions were then mixed and stirred at 600 rpm and at room temperature for 30 minutes. Finally, the clear, slightly amber-coloured solution was dried in a dry location protected from light. After pulverization, the solid dispersion was stored airtight and protected from light.

Common Solvent Evaporation 2:1 (CSE 2:1)

2000 mg Eudragit® E100 was weighed out and dissolved in 30 ml ethanol. 1000 mg taxifolin was then weighed out and dissolved in 15 ml ethanol. The two solutions were then mixed and stirred at 600 rpm and at room temperature for 30 minutes. Finally, the clear, slightly amber-coloured solution was dried in a dry location protected from light. After pulverization, the solid dispersion was stored airtight and protected from light.

Common Solvent Evaporation 3:1 (CSE 3:1)

3000 mg Eudragit® E100 was weighed out and dissolved in 35 ml ethanol. 1000 mg taxifolin was then weighed out and dissolved in 15 ml ethanol. The two solutions were then mixed and stirred at 600 rpm and at room temperature for 30 minutes. Finally, the clear, slightly amber-coloured solution was dried in a dry location protected from light. After pulverization, the solid dispersion was stored airtight and protected from light.

Specification of the Solid Dispersion

The solid dispersion was slightly amber in colour, glass-like, very hard/splintery, and free-flowing after pulverization. Under the light microscope, no recrystallization of the flavonoid was detected in any of the samples (1:1, 2:1, 3:1).

Yield: An important point, especially for scaling-up the process, is the yield of each of the methods. This allows the most efficient and thus the most cost-effective method to be selected.

| Solid dispersion | Yield (%) | Taxifolin content |
|---|---|---|
| CSE 1:1 | 82.3% | 50% |
| CSE 2:1 | 78.4% | 33.3% |
| CSE 3:1 | 87.9% | 25% |

The yields are in the range of 80%-90%; this is customary for the production of solid dispersions on a laboratory scale. On an industrial scale, the yield can be significantly increased by established methods such as continuous hot melt extrusion (HME) or spray drying.

DSC Analyses

DSC analysis is an important method to further characterize the solid dispersions.

Here, attention must be paid to both the glass transition temperature Tg of the polymer and the characteristic active ingredient peak. If both Tg and the active ingredient peak can be seen, the active ingredient is only finely distributed, but crystalline in the form of a solid suspension in the polymer. However, if the active ingredient peak disappears and only Tg can be found, the active ingredient is amorphous in the form of a solid solution in the polymer. Solid solutions usually have a better dissolution behaviour than solid suspensions and are to be preferred over the latter.

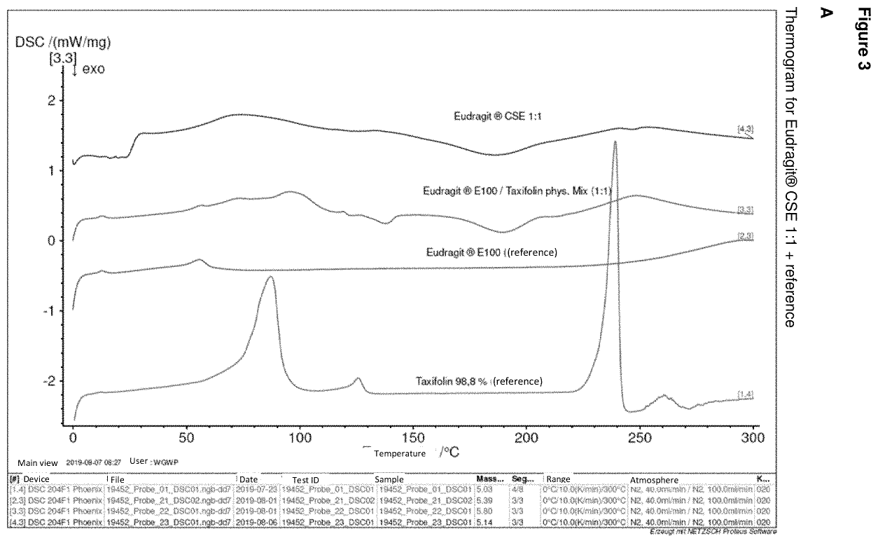

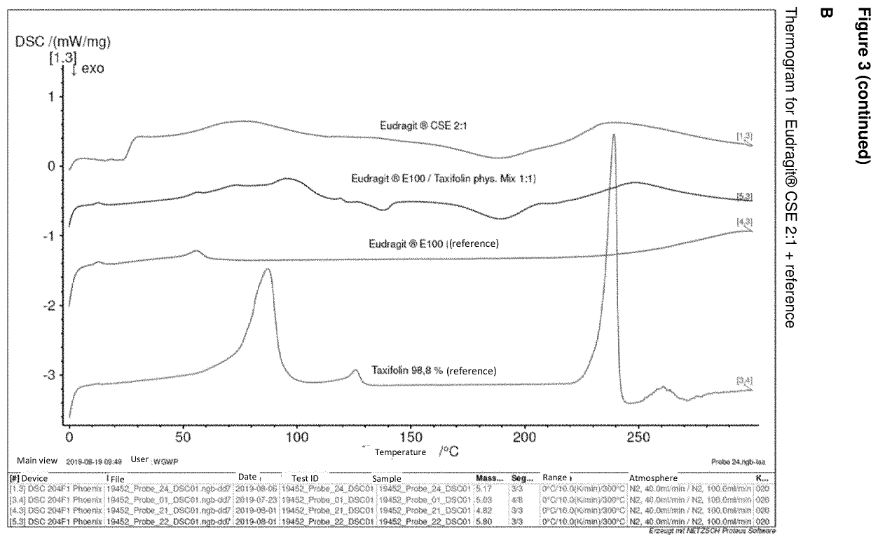

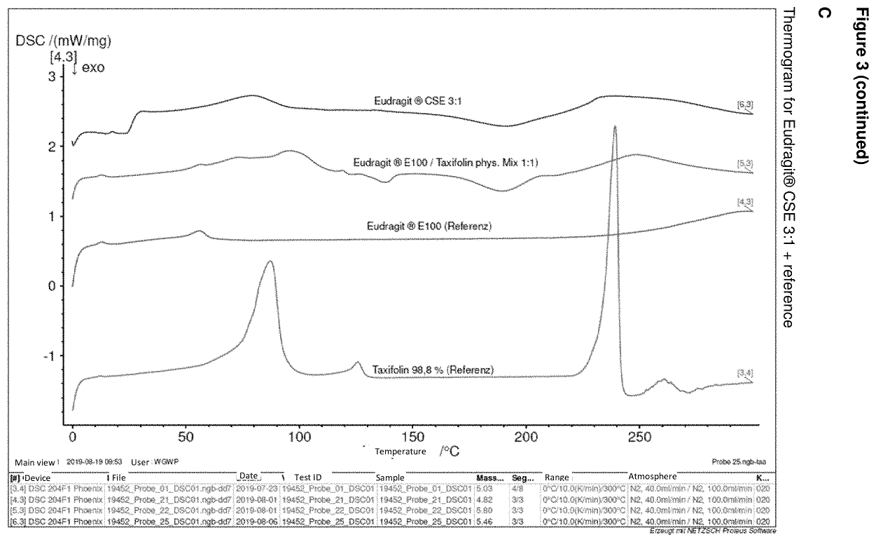

The results are shown in FIG. 3.

Discussion: The reference samples of the polymer and the flavonoid behave as expected. The polymer demonstrates an endothermic peak at 50° C., which is due to the melting of the polymer. Taxifolin demonstrates a sharp, characteristic endothermic peak at 239.2° C. In addition, a broad peak around 70° C.-100° C. in the taxifolin test indicates the escape of residual solvent.

The physical mixture demonstrates a 50° endothermic peak for the melting of the polymer. Moreover, a broader peak around 100° C. can be seen, which is probably due to the escape of residual water from the sample, similar to the taxifolin sample. The exothermic peaks in the range between 110° C.-210° C. are due to the dissolution of the crystalline taxifolin in the molten polymer. The ionic interactions and hydrogen bonds that develop between polymer and flavonoid stabilize the flavonoid in the amorphous state, which is why the characteristic active ingredient peak of the taxifolin also disappears in the phys. mixture. This also indicates that melt extrusion is a suitable method for producing a glass-like solid solution of taxifolin or similar flavonoids in basic polymethacrylates.

The three samples CSE 1:1, CSE 2:1, CSE 3:1 exhibit almost identical behaviour. The broad endothermic peak between 60° C. and 90° C. indicates the escape of residual solvent, in this case ethanol. The endothermic peak at 50° C. for the melting of the polymer disappears, and no more characteristic active ingredient peak can be seen. These points indicate the presence of a glassy solid solution in the three samples.

XRD Analysis

The XRD method is the method of choice to demonstrate complete, amorphous embedding of an active ingredient in the polymer matrix. The crystallinity of the sample is determined, which provides information on the arrangement of the active ingredient molecules. Since, in contrast to the active ingredient, the polymer matrix is amorphous, crystalline peaks indicate incomplete embedding. On the other hand, if the sample is amorphous, the solution is solid.

In addition, amorphous samples usually exhibit significantly better dissolution behaviour than crystalline samples, which is why an increase in bioavailability is possible with an amorphous sample.

Result: The diffraction diagrams demonstrate that both taxifolin and the phys. mixture of taxifolin/Eudragit® E100 are crystalline. As expected, the polymer is amorphous. The phys. mixture also shows superimposed X-ray diffraction patterns of taxifolin and Eudragit® E100. Moreover, all three formulations are amorphous and do not differ from the reference polymer.

Discussion: The results of the XRD analyses indicate that there are solid dispersions at CSE 1:1, CSE 2:1, and CSE 3:1, with the flavonoid taxifolin being completely embedded in the polymer matrix.

It is particularly interesting that a completely amorphous solid dispersion can be formulated even with a 1:1 polymer:flavonoid ratio. This is where basic polymethacrylates differ from most other polymers, in which a significantly higher proportion of polymer is necessary to formulate solid dispersions with taxifolin or similar flavonoids.

4. Solubility of the Solid Dispersion with Eudragit E

The last most important point for comparing the production methods to one another is solubility in simulated gastric juices. That is, the solubility of the complex directly influences bioavailability, because only dissolved active ingredients can pass the epithelial cells of the GI tract

Reference Measurement (Taxifolin)

10 mg taxifolin (Lavitol® 98.9% purity) was placed in a vial with 5 ml 0.1 N HCl to make a saturated solution and shaken for 60 minutes. The solution was then transferred into a vial by means of a syringe with an HPLC filter (0.22 μm) and then measured.

Sample Measurement

Saturated solutions of the samples in 0.1 molar HCl solution were prepared at room temperature. The solution was then transferred to a vial by means of a syringe with an HPLC filter (0.22 μm), appropriately diluted and the taxifolin concentration of the solution was determined by HLPC.

| | Weighed | Dilution | Solubility of |
|---|---|---|---|
| Name | in mg | factor | taxifolin in mg/ml |
| Taxifolin reference | 10.34 | — | 0.6927 |
| CSE 1:1 | 1034.06 | 80 | 12.04 |
| CSE 2:1 | 1331.63 | 66.6 | 15.00 |
| CSE 3:1 | 1516.45 | 50 | 10.13 |

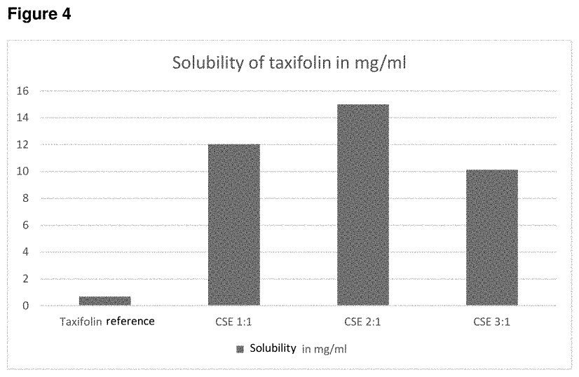

The results are shown in FIG. 4.

Discussion: By formulating a solid dispersion with basic polymethacrylates, it is possible to significantly increase the saturation solubility of taxifolin and probably also other flavonoids. This is due in particular to the fact that the flavonoid in all three formulations is embedded in the polymer in amorphous form, which is confirmed by both DSC and XRD analyses.

However, there are differences between the various amounts of polymer, with a polymer:flavonoid ratio of 2:1 achieving the best results in terms of saturation solubility. With regard to the amount of polymer used, an inverted U-shaped relationship can be seen with regard to the solubility.

If the amount of polymer is below the optimum, the ionic interaction between the flavonoid and the polymer means that there are not enough free, protonatable tertiary amino groups of the polymer, which reduces the solubility of the solid dispersion in water. If the amount of polymer is too high, the limiting factor is the protonation of the polymer, which, due to the interaction between the flavonoid and the polymer, has a retarding or inhibiting effect on the dissolution of the flavonoid.

It is therefore important to find the optimal ratio between polymer and flavonoid, with a ratio of 2:1 still appearing to be optimal.

Due to the small amount of polymer, the solid dispersion is ideal for formulating various pharmaceutical dosage forms. In addition, the special properties of the polymer result in other advantages, such as, for example, taste masking.

Discussion of Solid Dispersions of Basic Polymethacrylates

In general, solid dispersions are the gold standard for improving solubility, especially in the pharmaceutical sector. After polymer screening, basic polymethacrylates could be identified as suitable carriers and solid solutions could be formulated with them. In addition to Eudragit®, other basic polymethacrylates (Eudraguard® protect, Kollicoat® Smartseal, etc.) are also suitable for formulating solid dispersions with flavonoids such as taxifolin.

It is interesting here that, in addition to hydrogen bonds, there are also ionic interactions, and this made it possible to stabilize the flavonoid in its amorphous form particularly well. This was also reflected in the low polymer:flavonoid ratio, wherein the recrystallization of taxifolin could be effectively prevented with a ratio of up to 1:1, which DSC and XRD analyses confirm. This represents an enormous improvement over typical polymers such as PVP and PEG, which only effectively stabilized the flavonoid starting from a ratio of 9:1.

In addition, it was possible to demonstrate a significant increase in water solubility and an improvement in the dissolution behaviour, allowing an instant release formulation. Due to the small amount of polymer required, higher doses of taxifoline can also be taken with a small number of capsules/tablets, which increases compliance. This makes basic polymethacrylates the ideal carrier polymers for formulating solid dispersions with various flavonoids and thereby increasing bioavailability.

On an industrial scale, production can take place either using spray drying or hot-melt extrusion (melt extrusion), although other production methods are also conceivable.

### 5. Dissolution Behaviour Formulations

In order to check the dissolution behaviour of the final formulations, dissolution studies of the cyclodextrin and the Eudragit formulation against pure taxifolin were carried out. It is to be expected that the instant-release formulations significantly improve the dissolution behaviour of the flavonoid, since the pure taxifolin only dissolves very slowly due to the stable crystalline structure and the low solubility in water.

The solid dispersion with Eudragit® E dissolves the crystalline structure (see XRPD analyses) and thus increases solubility in water. In the CD complexes, the crystalline structure is also dissolved by encapsulating each individual taxifolin molecule; at the same time, the CD, as a “Trojan horse”, increases water solubility and wettability. Both should lead to an improvement in the dissolution behaviour.

The instant-release formulation is considered optimal if 85% of the active ingredient has dissolved in the first 15 minutes. Since gastric emptying when fasting is a reaction of the first order (50% emptying in 10-20 min), with 85% dissolution in the first 15 min, it can be assumed that the formulation behaves like a solution and therefore optimally.

Method: In order to determine the dissolution behaviour, the usual procedure according to pharmacopoeia was chosen.

USP apparatus II (paddle); 100 rpm; medium: 500 ml 0.1 N HCl; 2 vessels per sample (N=2); 7 sampling points: 0 min, 5 min, 10 min, 15 min, 20 min, 30 min, 60 min; weighed in: formulation as a powder corresponding to 100 mg taxifolin; detection by HPLC

The following formulations were tested:
taxifolin (Ametis Lavitol®, 98.8% purity)
Eudragit® E CSE 2:1 (solid dispersion formulation)
FD β (cyclodextrin formulation)

The pure taxifolin is the reference value.

The CSE 2:1 was chosen as the formulation for the solid dispersion, since with this ratio of polymer:taxifolin recrystallization of the flavonoid can be ruled out and the flavonoid is completely embedded amorphously in the polymer matrix, which DSC and XRD analyses confirm. Moreover, this formulation achieved the maximum saturation solubility and is therefore ideally suited for dissolution tests.

The FD β complex was chosen as the cyclodextrin formulation because the freeze-dry method is considered the gold standard for producing various cyclodextrin complexes in research, and moreover this method also achieved optimal results in terms of encapsulation efficiency and saturation solubility. Although the method is very cost-intensive and difficult to scale, it is still ideally suited for tests on a laboratory scale. This is also due to the fact that lyophilized complexes usually have good dissolution behaviour due to the small particle size and the high surface area. In addition, this method is very gentle due to the low temperatures, which means that product degradation can be ruled out.

Results:

Release of taxifolin (reference)

| | | | | |
|---|---|---|---|---|
| | | Weighed | | Mean |
| Test time | Vessel | in mg | Release | release |

| | | | | | |
|---|---|---|---|---|---|
| 5 | min | 1 | 106.52 | 28.2% | 29% |
| | | 2 | 108.20 | 29.2% | |
| 10 | min | 1 | 106.52 | 46.4% | 46% |
| | | 2 | 108.20 | 44.8% | |
| 15 | min | 1 | 106.52 | 61.1% | 60% |
| | | 2 | 108.20 | 58.7% | |
| 20 | min | 1 | 106.52 | 70.4% | 69% |
| | | 2 | 108.20 | 67.5% | |
| 30 | min | 1 | 106.52 | 80.8% | 79% |
| | | 2 | 108.20 | 77.4% | |
| 60 | min | 1 | 106.52 | 90.8% | 92% |
| | | 2 | 108.20 | 93.1% | |

Release of taxifolin/β-CD complex FD β

| | | | | |
|---|---|---|---|---|
| | | Weighed | | Mean |
| Test time | Vessel | in mg | Release | release |

| | | | | | |
|---|---|---|---|---|---|
| 5 | min | 1 | 586.93 | 100.1% | 100% |
| | | 2 | 587.81 | 100.5% | |
| 10 | min | 1 | 586.93 | 100.6% | 100% |
| | | 2 | 587.81 | 100.2% | |
| 15 | min | 1 | 586.93 | 103.2% | 103% |
| | | 2 | 587.81 | 103.1% | |
| 20 | min | 1 | 586.93 | 100.1% | 100% |
| | | 2 | 587.81 | 100.5% | |
| 30 | min | 1 | 586.93 | 100.5% | 101% |
| | | 2 | 587.81 | 101.1% | |
| 60 | min | 1 | 586.93 | 100.3% | 101% |
| | | 2 | 587.81 | 101.3% | |

Release of Eudragit ® E CSE 2:1

| | | | | |
|---|---|---|---|---|
| | | Weighed | | Mean |
| Test time | Vessel | in mg | Release | release |

| | | | | | |
|---|---|---|---|---|---|
| 5 | min | 1 | 301.21 | 82.2% | 82.2% |
| | | 2 | 303.27 | (150.0%) | |
| 10 | min | 1 | 301.21 | 84.8% | 85% |
| | | 2 | 303.27 | 84.5% | |
| 15 | min | 1 | 301.21 | 86.6% | 86% |
| | | 2 | 303.27 | 86.2% | |
| 20 | min | 1 | 301.21 | 85.5% | 85% |
| | | 2 | 303.27 | 84.4% | |
| 30 | min | 1 | 301.21 | 85.3% | 85% |
| | | 2 | 303.27 | 84.9% | |
| 60 | min | 1 | 301.21 | 85.0% | 85% |
| | | 2 | 303.27 | 84.8% | |

Note:
At the test time of 5 minutes, vessel 2, a particle was drawn through the filter, which had dissolved before the measurement. This measurement point was therefore not included.

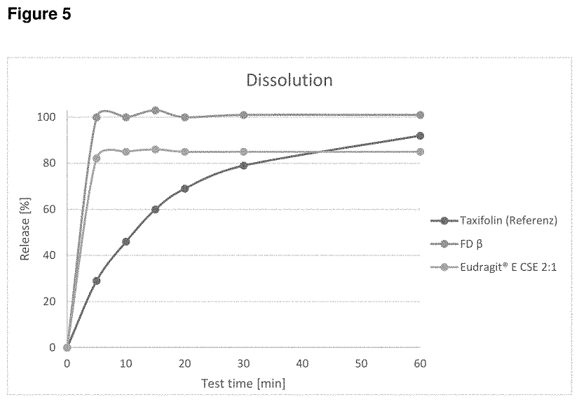

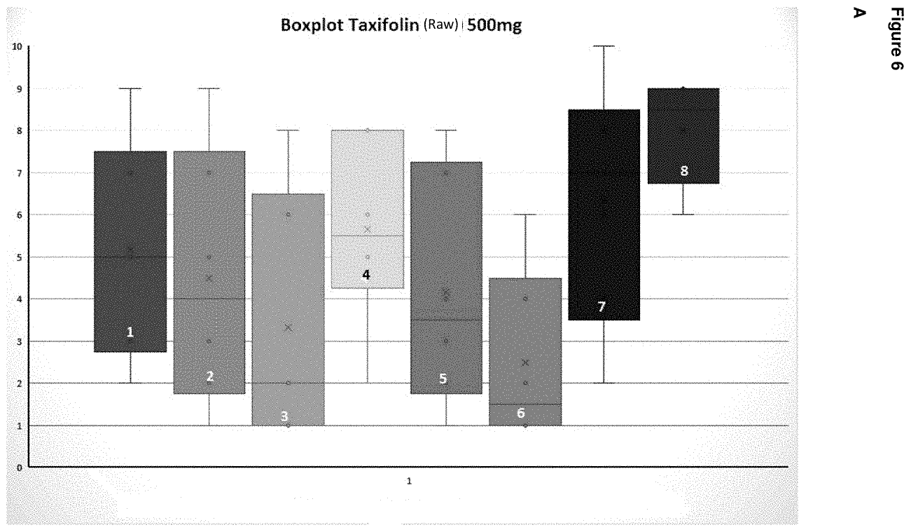

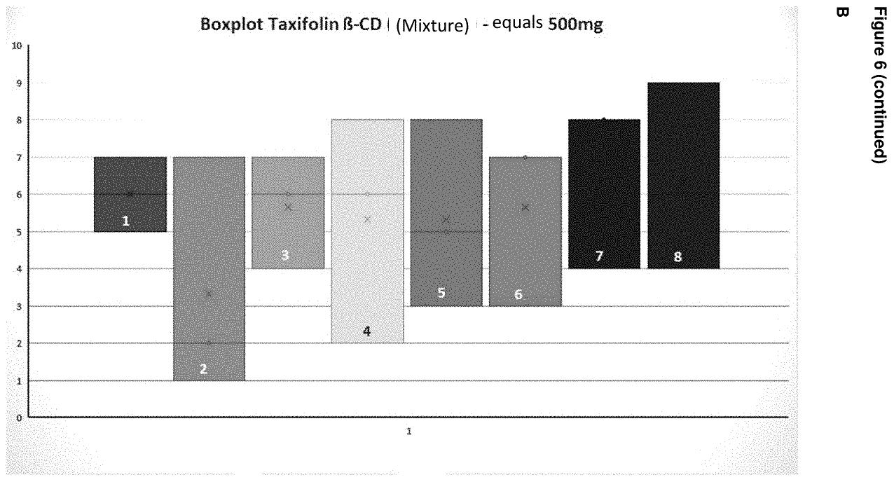

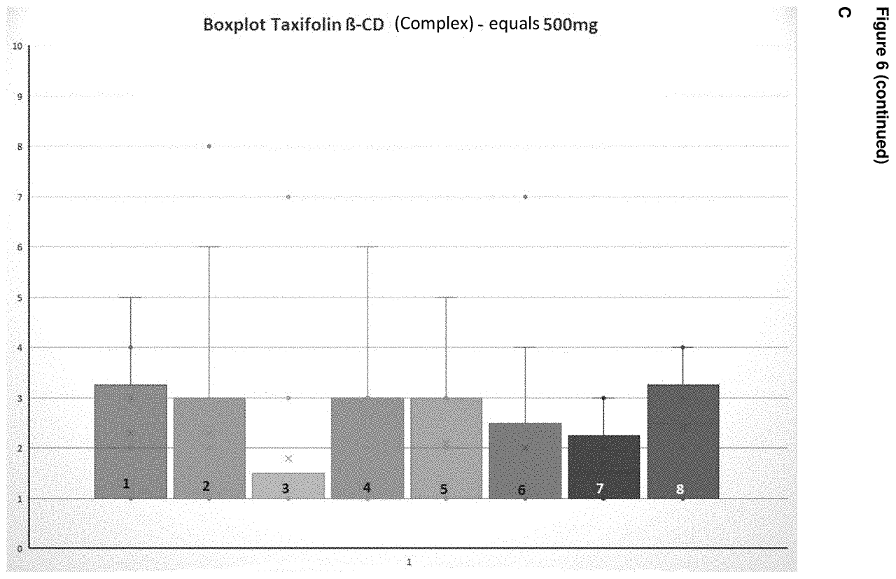

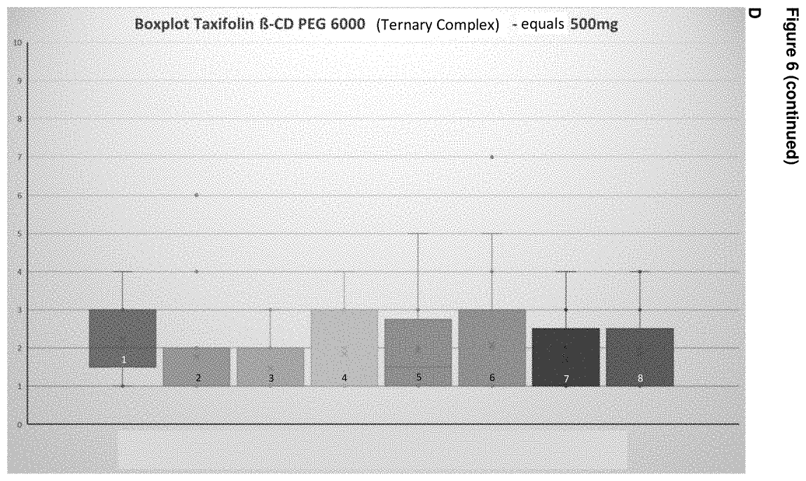

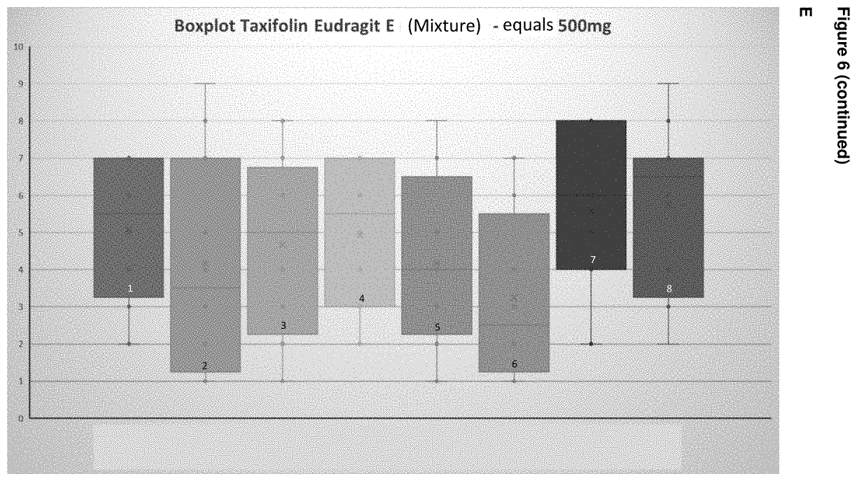

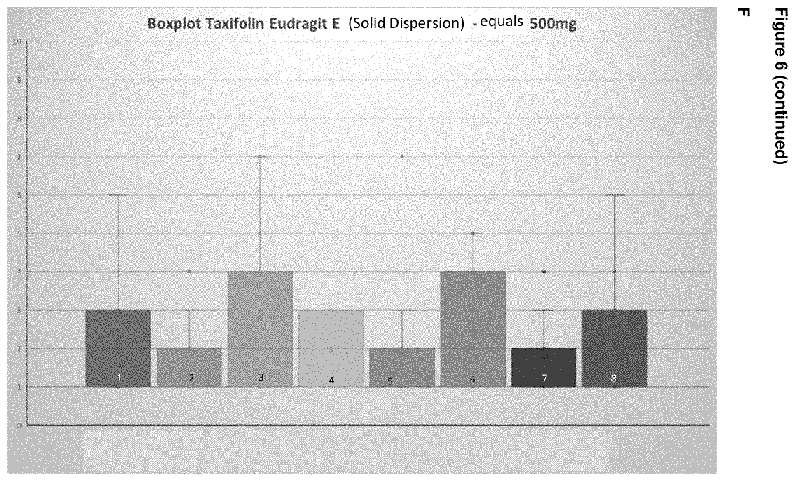

The results are shown in FIG. 5.

Discussion: In free form, taxifolin demonstrates typical dissolution behaviour with continuous release. However, the release after 15 minutes is only 60% and therefore does not meet the requirement of an instant-release formulation (min. 85% after 15 minutes). This means that the dissolution behaviour and thus the bioavailability of the flavonoid can in principle be improved using a suitable formulation. Both the solid dispersion in Eudragit® E and the cyclodextrin formulation FD β meet the requirements and are therefore considered to be optimal instant-release formulations.

FD β releases the flavonoid very quickly and has already achieved 100% release at the first measurement point. In addition, there is no recrystallization in the sense of a “spring-parachute effect”, instead the release is always 100%.

The Eudragit® E formulation also achieves a very rapid release of the flavonoid, with 82.2% of the flavonoid already in solution at the first measurement point. Here, too, there is no recrystallization and the taxifolin does not precipitates out of the solution, but the release of the taxifolin is limited to a maximum of 85%. This was also demonstrated by the fact that residues of the solid dispersion were still to be found in the vessel after the 60 minutes had elapsed.

This behaviour could also be observed in various preliminary tests with other polymers, wherein mostly a residue of the solid dispersion with taxifolin remained and was difficult to dissolve, especially with a low polymer:flavonoid ratio. This is due to the fact that the flavonoid competes with the solvent water for interactions with the functional groups of the polymer. Basic polymethacrylates have protonatable tertiary amine groups, which significantly increase the water solubility of the solid dispersion, even with a low polymer content, but there may be competition between the flavonoid and the solvent. Under certain circumstances, this problem can be solved by increasing the polymer content, but this will reduce the saturation solubility and there may be a delay in the release of flavonoid. However, this must be evaluated in further series of tests.

In conclusion, it may be said that both the formulation of a solid dispersion with basic polymethacrylates such as Eudragit® E and an inclusion complex with β-CD can greatly improve the dissolution behaviour of the flavonoid taxifolin. Both formulations also satisfy the requirements as an instant-release formulation and are therefore in principle suitable for increasing the bioavailability of various flavonoids.

### 6. Investigation of Bioavailability

The bioavailability of taxifolin and its formulations when administered to humans was investigated using efficacy studies. These were single-blind studies (subject blind) conducted on ten healthy volunteers from the University of Regensburg. Each test person took a total of six different preparations.

Each test preparation was a formulation containing a total of 500 mg taxifolin, administered either as pure taxifolin (Lavitol 99.8%), as an equimolar β-CD/taxifolin mixture (1:1 physical mixture), as a β-CD/taxifolin complex (FD β), as a β-CD/taxifolin/PEG6000 ternary complex (FD β+80 mg PEG 6000), as a Eudragit®E/taxifolin mixture in a weight ratio of 2:1, or as a solid dispersion of Eudragit®E/taxifolin (CSE 2:1). The taxifolin-containing formulations were first weighed out and mixed with the correspondingly calculated amount of filler (microcrystalline cellulose). Finally, the formulations were filled into size 0 gelatin capsules.

In order to avoid falsification of the results, before taking the formulations the test subjects followed a one-week wash-out phase in which they were not to consume alcohol or tobacco products. Before taking the test preparation, each test subject consumed 1.5 g ethanol per kilogram of body weight, in the form of vodka with 37.5% ethanol content, spread over 4 hours. Ten hours after taking the preparation, eight typical hangover symptoms were evaluated using a questionnaire. The test subjects rated the symptoms on a scale of 1-10, with 1 meaning no symptoms and 10 meaning very strong symptoms. The test series shown in FIG. 6 A-F show tests with pure taxifolin (FIG. 6A), with beta-cyclodextrin as a mixture (FIG. 6B), and as a complex with beta-cyclodextrin (FIG. 6C), with a ternary complex taxifolin/3-CD/PEG6000 (FIG. 6D), with Eudragit E as a mixture (FIG. 6E), and with Eudragit E in the form of a solid dispersion (FIG. 6F). The following symptoms were investigated: (1) General condition, (2) Headache, (3) Nausea, (4) Dizziness, (5) Cognitive performance, (6) Gastrointestinal, (7) Motivation, and (8) Fatigue.

Discussion: The results clearly demonstrate that the bioavailability and thus the effectiveness of taxifolin can be significantly improved by formulating it with cyclodextrins or basic polymethacrylates.

Even the mixture with cyclodextrin improves bioavailability, but significantly better results were achieved with the cyclodextrin complex, which is most likely due to the increase in the solubility and stability of the flavinoid. However, the best results were achieved by combining the cyclodextrin complex with the water-soluble polymer PEG 6000. This can be explained by the formation of a ternary complex, which significantly prevents the formation of supramolecular cyclodextrin complex aggregates and, moreover, increased the complex stability.

The mixture of taxifolin and Eudragit E also achieved an improvement in efficacy compared to pure taxifolin. However, the solid dispersion proved to be more effective compared to the mixture, which can be attributed to the amorphous distribution of the flavonoid in the polymer matrix and the associated improvement in solubility.

### REFERENCES

1. Martin Wallner, H. J. Hanchar, R. W. Olsen; Ethanol enhances α4β3δ and α6β3δ GABAA receptors at low concentrations known to affect humans; Proc. Natl. Acad. Sci. 2003, 100 (25): 15218-15223.
2. M. Wallner, H. J. Hanchar, and R. W. Olsen; Low-dose alcohol actions on α4β3δ GABAA receptors are reversed by the behavioral alcohol antagonist Ro15-4513; Proc. Natl. Acad. Sci. USA. 2006 May 30; 103 (22): 8540-8545.
3. Wallner M., Hanchar H. J., Olsen R. W.; Alcohol selectivity of β3-containing GABAA receptors: evidence for a unique extracellular alcohol/imidazobenzodiazepine Ro15-4513 binding site at the α+β-subunit interface in αβ3δ GABAA receptors; Neurochem Res. 2014 June; 39 (6): 1118-26.
4. Hammer H., Bader B. M., Ehnert C., Bundgaard C., Bunch L., Hoestgaard-Jensen K., Schroeder O. H., Bastlund J. F., Gramowski-Voss A., Jensen A. A.; A Multifaceted GABAA Receptor Modulator: Functional Properties and Mechanism of Action of the Sedative-Hypnotic and Recreational Drug Methaqualone (Quaalude).; Mol pharmacol. 2015 August; 88 (2):401-20.
5. Juan Chen, Yang He, Yan Wu, Hang Zhou, Li-Da Su, Wei-Nan Li, Richard W. Olsen, Jing Liang, Yu-Dong Zhou, and Yi Shen; Single Ethanol Withdrawal Regulates Extrasynaptic δ-GABAA Receptors Via PKCδ Activation; Front Mol Neurosci. 2018 11 141.
6. József Nagy; Alcohol Related Changes in Regulation of NMDA Receptor Functions; Curr Neuropharmacol. 2008 March; 6 (1): 39-54.

1-21. (canceled)

22. A flavonoid of the general formula (I) 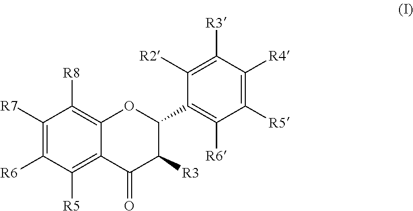
wherein:
R2′, R5′, R6′, R6, and R8 are each —H, and,
R3′, R4′, R3, R5, and R7 are each —OH.
wherein the flavonoid
(i) is present as a complex of the general formula (II) 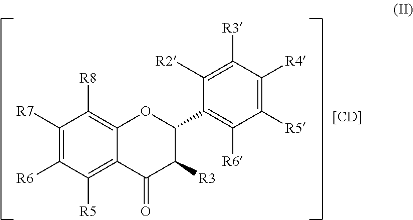
wherein CD is a β-cyclodextrin molecule, which can be unsubstituted or substituted on one or more hydroxyl groups, or
(ii) is a solid dispersion with a basic (co)polymer of methacrylic acid and/or methacrylate.

23. The flavonoid according to claim 22, wherein said β-cyclodextrin molecule is substituted on the C6 carbon atom of one or more glucose units.

24. The flavonoid according to claim 22, wherein said basic (co)polymer of methacrylic acid and/or methacrylate is Eudragit®E (cationic copolymer based on dimethylaminoethyl methacrylate).

25. The flavonoid according to claim 22, wherein CD is a β-cyclodextrin, which is substituted on one or more hydroxyl groups with —O—C₁₋₁₈-alkyl or —O—C₁₋₁₈-hydroxyalkyl groups.

26. The flavonoid according to claim 25, wherein CD is a β-cyclodextrin, which is substituted on the C6 carbon atom of one or more glucose units.

27. The flavonoid according to claim 22, wherein the flavonoid is a complex of the general formula (II), and wherein the complex further comprises a water-soluble polymer.

28. The flavonoid according to claim 27, wherein said water soluble polymer is selected from the group consisting of polyethylene glycol, polyvinyl alcohol, poloxamer, and mixtures thereof.

29. A pharmaceutical composition for oral administration comprising a flavonoid according to claim 22, wherein said flavonoid is
a) a flavonoid complex of the general formula (II),
b) a flavonoid complex of the general formula (II) wherein the complex further comprises a water-soluble polymer, or
c) a flavonoid of the general formula (I) wherein said flavonoid is a solid dispersion with a basic (co)polymer of methacrylic acid and/or methacrylate.

30. The pharmaceutical composition according to claim 29, further comprising one or more pharmaceutically acceptable adjuvants and/or excipients suitable for oral administration.

31. A method for inhibiting and/or treating alcoholism, alcohol intoxication, and consequential symptoms associated with alcohol consumption, or reducing consequential diseases associated with alcohol consumption, comprising administering a flavonoid of the general formula (I) to a patient in need of such treatment, 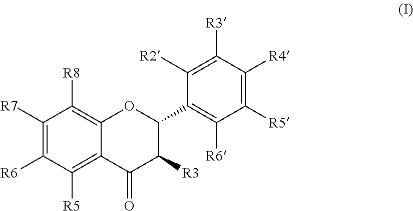
wherein:
R2′, R5′, R6′, R6, and R8 are each —H, and,
R3′, R4′, R3, R5, and R7 are each —OH.
wherein the flavonoid
(i) is present as a complex of the general formula (II) 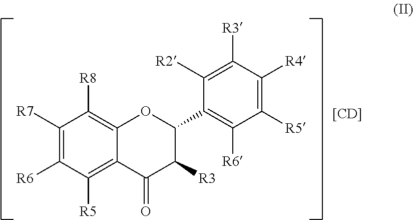
wherein CD is a β-cyclodextrin molecule, which can be unsubstituted or substituted on one or more hydroxyl groups, or
(ii) is a solid dispersion with a basic (co)polymer of methacrylic acid and/or methacrylate.

32. The method according to claim 31, wherein said β-cyclodextrin molecule is substituted on the C6 carbon atom of one or more glucose units.

33. The method according to claim 31, wherein said basic (co)polymer of methacrylic acid and/or methacrylate is Eudragit®E (cationic copolymer based on dimethylaminoethyl methacrylate).

34. The method according to claim 31, wherein CD is a β-cyclodextrin, which is substituted on one or more hydroxyl groups with —O—C₁₋₁₈-alkyl or —O—C₁₋₁₈-hydroxyalkyl groups.

35. The method according to claim 34, wherein CD is a β-cyclodextrin, which is substituted on the C6 carbon atom of one or more glucose units.

36. The method according to claim 31, wherein the flavonoid is a complex of the general formula (II), and wherein the complex further comprises a water-soluble polymer.

37. The method according to claim 36, wherein said water soluble polymer is selected from the group consisting of polyethylene glycol, polyvinyl alcohol, poloxamer, and mixtures thereof.

38. The method according to claim 31, wherein said consequential symptoms associated with alcohol consumption comprise hangover symptoms.

39. The method according to claim 31, wherein consequential symptoms and diseases associated with alcohol consumption comprise neurological damage due to alcohol intoxication.

40. The method according to claim 31, wherein the treatment of alcoholism comprises alcohol dehabituation and/or alcohol withdrawal.
